SEQUENCE LISTING (72 sequences)

SEQ ID NO: 1 - protein, 258 aa, human
MLPLCLVAALLLAAGPGPSLGDEAIHCPPCSEEKLARCRPPVGCEELVREPGCGCCATCALGLGMPCGVYTPRCGSGLRCYPPRGVEKPLHTLMHGQGVCMELAEIEAIQESLQPSDKDEGDHPNNSFSPCSAHDRRCLQKHFAKIRDRSTSGGKMKVNGAPREDARPVPQGSCQSELHRALERLAASQSRTHEDLYIIPIPNCDRNGNFHPKQCHPALDGQRGKCWCVDRKTGVKLPGGLEPKGELDCHQLADSFRE

SEQ ID NO: 2 - protein, 282 aa, artificial
  IGFBP-4 construct
MLPLCLVAALLLAAGPGPSLGDEAIHCPPCSEEKLARCRPPVGCEELVREPGCGCCATCALGLGMPCGVYTPRCGSGLRCYPPRGVEKPLHTLMHGQGVCMELAEIEAIQESLQPSDKDEGDHPNNSFSPCSAHDRRCLQKHFAKIRDRSTSGGKMKVNGAPREDARPVPQGSCQSELHRALERLAASQSRTHEDLYIIPIPNCDRNGNFHPKQCHPALDGQRGKCWCVDRKTGVKLPGGLEPKGELDCHQLADSFREVDPWSHPQFEKTGHHHHHHHHGGQ

SEQ ID NO: 3 - protein, 179 aa, artificial
  IGFBP4 N-terminal fragment construct
MLPLCLVAALLLAAGPGPSLGDEAIHCPPCSEEKLARCRPPVGCEELVREPGCGCCATCALGLGMPCGVYTPRCGSGLRCYPPRGVEKPLHTLMHGQGVCMELAEIEAIQESLQPSDKDEGDHPNNSFSPCSAHDRRCLQKHFAKIRDRSTSGGKMDPWSHPQFEKTGHHHHHHHHGGQ

SEQ ID NO: 4 - protein, 146 aa, artificial
  IGFBP4 C-terminal construct
MLLLLLLLGLRLQLSLGIASKVNGAPREDARPVPQGSCQSELHRALERLAASQSRTHEDLYIIPIPNCDRNGNFHPKQCHPALDGQRGKCWCVDRKTGVKLPGGLEPKGELDCHQLADSFREVDPWSHPQFEKTGHHHHHHHHGGQ

SEQ ID NO: 5 - protein, 31 aa, human
PIPNCDRNGNFHPKQCHPALDGQRGKCWCVD

SEQ ID NO: 6 - protein, 237 aa, human
DEAIHCPPCSEEKLARCRPPVGCEELVREPGCGCCATCALGLGMPCGVYTPRCGSGLRCYPPRGVEKPLHTLMHGQGVCMELAEIEAIQESLQPSDKDEGDHPNNSFSPCSAHDRRCLQKHFAKIRDRSTSGGKMKVNGAPREDARPVPQGSCQSELHRALERLAASQSRTHEDLYIIPIPNCDRNGNFHPKQCHPALDGQRGKCWCVDRKTGVKLPGGLEPKGELDCHQLADSFRE

SEQ ID NO: 7 - protein, 135 aa, human
DEAIHCPPCSEEKLARCRPPVGCEELVREPGCGCCATCALGLGMPCGVYTPRCGSGLRCYPPRGVEKPLHTLMHGQGVCMELAEIEAIQESLQPSDKDEGDHPNNSFSPCSAHDRRCLQKHFAKIRDRSTSGGKM

SEQ ID NO: 8 - protein, 102 aa, human
KVNGAPREDARPVPQGSCQSELHRALERLAASQSRTHEDLYIIPIPNCDRNGNFHPKQCHPALDGQRGKCWCVDRKTGVKLPGGLEPKGELDCHQLADSFRE

SEQ ID NO: 9 - protein, 20 aa, human
DEAIHCPPCSEEKLARCRPP

SEQ ID NO: 10 - protein, 20 aa, human
EEKLARCRPPVGCEELVREP

SEQ ID NO: 11 - protein, 20 aa, human
PVGCEELVREPGCGCCATCA

SEQ ID NO: 12 - protein, 20 aa, human
PGCGCCATCALGLGMPCGVY

SEQ ID NO: 13 - protein, 20 aa, human
ALGLGMPCGVYTPRCGSGLR

SEQ ID NO: 14 - protein, 20 aa, human
YTPRCGSGLRCYPPRGVEKP

SEQ ID NO: 15 - protein, 20 aa, human
RCYPPRGVEKPLHTLMHGQG

SEQ ID NO: 16 - protein, 20 aa, human
PLHTLMHGQGVCMELAEIEA

SEQ ID NO: 17 - protein, 20 aa, human
VCMELAEIEAIQESLQPSDK

SEQ ID NO: 18 - protein, 20 aa, human
AIQESLQPSDKDEGDHPNNS

SEQ ID NO: 19 - protein, 20 aa, human
KDEGDHPNNSFSPCSAHDRR

SEQ ID NO: 20 - protein, 20 aa, human
SFSPCSAHDRRCLQKHFAKI

SEQ ID NO: 21 - protein, 20 aa, human
RCLQKHFAKIRDRSTSGGKM

SEQ ID NO: 22 - protein, 20 aa, human
IRDRSTSGGKMKVNGAPRED

SEQ ID NO: 23 - protein, 20 aa, human
MKVNGAPREDARPVPQGSCQ

SEQ ID NO: 24 - protein, 20 aa, human
ARPVPQGSCQSELHRALERL

SEQ ID NO: 25 - protein, 20 aa, human
QSELHRALERLAASQSRTHE

SEQ ID NO: 26 - protein, 20 aa, human
LAASQSRTHEDLYIIPIPNC

SEQ ID NO: 27 - protein, 20 aa, human
EDLYIIPIPNCDRNGNFHPK

SEQ ID NO: 28 - protein, 20 aa, human
CDRNGNFHPKQCHPALDGQR

SEQ ID NO: 29 - protein, 20 aa, human
QCHPALDGQRGKCWCVDRKT

SEQ ID NO: 30 - protein, 20 aa, human
RGKCWCVDRKTGVKLPGGLE

SEQ ID NO: 31 - protein, 20 aa, human
RKTGVKLPGGLEPKGELDCH

SEQ ID NO: 32 - protein, 17 aa, human
EPKGELDCHQLADSFRE

SEQ ID NO: 33 - protein, 51 aa, human
KVNGAPREDARPVPQGSCQSELHRALERLAASQSRTHEDLYIIPIPNCDRN

SEQ ID NO: 34 - protein, 51 aa, human
GNFHPKQCHPALDGQRGKCWCVDRKTGVKLPGGLEPKGELDCHQLADSFRE

SEQ ID NO: 35 - protein, 17 aa, human
KVNGAPREDARPVPQGS

SEQ ID NO: 36 - protein, 17 aa, human
CQSELHRALERLAASQS

SEQ ID NO: 37 - protein, 17 aa, human
RTHEDLYIIPIPNCDRN

SEQ ID NO: 38 - protein, 17 aa, human
GNFHPKQCHPALDGQRG

SEQ ID NO: 39 - protein, 17 aa, human
KCWCVDRKTGVKLPGGL

SEQ ID NO: 40 - protein, 17 aa, human
EPKGELDCHQLADSFRE

SEQ ID NO: 41 - protein, 20 aa, human
GAPREDARPVPQGSCQSELH

SEQ ID NO: 42 - protein, 20 aa, human
REDARPVPQGSCQSELHRAL

SEQ ID NO: 43 - protein, 20 aa, human
RPVPQGSCQSELHRALERLA

SEQ ID NO: 44 - protein, 20 aa, human
PQGSCQSELHRALERLAASQ

SEQ ID NO: 45 - protein, 20 aa, human
SCQSELHRALERLAASQSRT

SEQ ID NO: 46 - protein, 21 aa, human
SELHRALERLAASQSRTHEDL

SEQ ID NO: 47 - protein, 21 aa, human
HRALERLAASQSRTHEDLYII

SEQ ID NO: 48 - protein, 21 aa, human
LERLAASQSRTHEDLYIIPIP

SEQ ID NO: 49 - protein, 21 aa, human
LAASQSRTHEDLYIIPIPNCD

SEQ ID NO: 50 - protein, 21 aa, human
SQSRTHEDLYIIPIPNCDRNG

SEQ ID NO: 51 - protein, 21 aa, human
RTHEDLYIIPIPNCDRNGNFH

SEQ ID NO: 52 - protein, 21 aa, human
EDLYIIPIPNCDRNGNFHPKQ

SEQ ID NO: 53 - protein, 21 aa, human
YIIPIPNCDRNGNFHPKQCHP

SEQ ID NO: 54 - protein, 21 aa, human
PIPNCDRNGNFHPKQCHPALD

SEQ ID NO: 55 - protein, 21 aa, human
PIPNCDRNGNFHPKQCHPALD

SEQ ID NO: 56 - protein, 21 aa, human
RNGNFHPKQCHPALDGQRGKC

SEQ ID NO: 57 - protein, 21 aa, human
NFHPKQCHPALDGQRGKCWCV

SEQ ID NO: 58 - protein, 21 aa, human
PKQCHPALDGQRGKCWCVDRK

SEQ ID NO: 59 - protein, 21 aa, human
CHPALDGQRGKCWCVDRKTGV

SEQ ID NO: 60 - protein, 21 aa, human
ALDGQRGKCWCVDRKTGVKLP

SEQ ID NO: 61 - protein, 21 aa, human
GQRGKCWCVDRKTGVKLPGGL

SEQ ID NO: 62 - protein, 21 aa, human
GKCWCVDRKTGVKLPGGLEPK

SEQ ID NO: 63 - protein, 21 aa, human
CWCVDRKTGVKLPGGLEPKGE

SEQ ID NO: 64 - protein, 21 aa, human
DRKTGVKLPGGLEPKGELDCH

SEQ ID NO: 65 - protein, 21 aa, human
TGVKLPGGLEPKGELDCHQLA

SEQ ID NO: 66 - protein, 21 aa, human
KLPGGLEPKGELDCHQLADSF

SEQ ID NO: 67 - protein, 21 aa, human
PGGLEPKGELDCHQLADSFRE

SEQ ID NO: 68 - DNA, 32 bp, artificial
  IGFBP4 forward primer for cloning
taagaattcgccaccatgctgcccctctgcct

SEQ ID NO: 69 - DNA, 30 bp, artificial
  IGFBP4 reverse primer for cloning
ttaggatccacctctcgaaagctgtcagcc

SEQ ID NO: 70 - DNA, 26 bp, artificial
  IGFBP primer for cloning n terminal fragment
ttaggatccatcttgcccccactggt

SEQ ID NO: 71 - DNA, 32 bp, artificial
  IGFBP primer for cloning c terminal fragment
gccgctagcaaggtcaatggggcgccccggga

SEQ ID NO: 72 - protein, 19 aa, artificial
  SEAP signal sequence
MLLLLLLLGLRLQLSLGIA

(19) United States
(12) Patent Application Publication
(10) Pub. No.: US 2009/0048158 A1
(43) Pub. Date: Feb. 19, 2009

(54) Insulin-Like Growth Factor Binding Protein-4 Compounds and Methods for Inhibiting Angiogenesis and Tumor Growth in Mammalian Cells

(76) Inventors: Maria J. Moreno, Ottawa (CA); Danica B. Stanimirovic, Ottawa (CA); Marguerite Ball, Ottawa (CA); Yves Durocher, Montreal (CA)

Correspondence Address: ADE & COMPANY INC., 2157 Henderson Highway, WINNIPEG, MB, R2G1P9, CA

(21) Appl. No.: 11/816,663

(22) PCT Filed: Feb. 20, 2006
(86) PCT No.: PCT/CA2006/000250

§ 371 Date: Jan. 11, 2008

Related U.S. Application Data
(60) Provisional application No. 60/653,958, filed on Feb. 18, 2005.

(51) Int. Cl. A61K 38/00

(52) U.S. Cl. 514/12

(57) ABSTRACT

The use of fragments of IGFBP-4 for inhibiting angiogenesis and tumor growth is described.

### PRIOR APPLICATION INFORMATION

This application claims the benefit of U.S. Provisional Patent application 60/653,958, filed Feb. 18, 2005.

### BACKGROUND OF THE INVENTION

Angiogenesis is critical for growth and progression of malignant tumours since proliferative cells are dependent on blood flow for nutrient and oxygen delivery. Disruption of tumor blood supply through inhibition of angiogenesis has emerged as an attractive strategy to control both tumor growth and metastasis. Preclinal studies using angiogenesis inhibitors showed partial or complete tumor regression without drug resistance (Kim et al., 1993; Ferrara, 2002). Clinical trials, however, have failed to repeat the success of preclinical studies due primarily to the multiple and synergistic angiogenesis pathways activated in late stage tumours (Cao, 2004). This underscores the need for more effective anti-angiogenic agents capable of counteracting angiogenic responses induced by the variety of growth factors produced during tumor progression.

Glioblastoma multiforme (GBM) is one of the most malignant and angiogenic of human tumors. The degree of GBM neovascularization directly correlates with an unfavorable prognosis. Malignant gliomas display lower cyclic adenosine 3′,5′-monophosphate (cAMP) content and reduced adenylate cyclase activity relative to normal brain tissue. Growth of malignant cells resulting from an imbalance of cAMP signal transducers can be inhibited with site-selective cAMP analogs. Evidence supporting the involvement of cAMP signaling pathways in the pathogenesis of glial tumors has promoted the use of cAMP analogs, alone or in combination with other cytostatic drugs, for suppression of tumor growth (Dalbasti et al., 2002; Propper et al., 1999). Despite observed delays in tumor growth and recurrence by these drugs, the involvement of cAMP in a multitude of signaling pathways relevant for cell physiology has restricted their systemic use for cancer therapy (Propper et al., 1999). The identification of adenylate cyclase-modulated downstream effectors is important to the discovery of more suitable and selective therapeutic targets for the treatment of gliomas.

### SUMMARY OF THE INVENTION

According to a first aspect of the invention, there is provided the use of a peptide comprising 20 or more consecutive amino acids of amino acids 1 to 258 of SEQ ID No. 1 in the preparation of a medicament for inhibiting angiogenesis or tumor growth.

According to a second aspect of the invention, there is provided the use of a peptide comprising at least 70% identity to amino acids 200-249 of SEQ ID No. 1 in the preparation of a medicament for inhibiting angiogenesis or tumor growth.

According to a third aspect of the invention, there is provided the use of a peptide comprising at least 70% identity to amino acids 1-155 of SEQ ID No. 1 in the preparation of a medicament for inhibiting angiogenesis or tumor growth.

According to a fourth aspect of the invention, there is provided the use of a peptide comprising at least 70% identity to amino acids 155-258 of SEQ ID No. 1 in the preparation of a medicament for inhibiting angiogenesis or tumor growth.

A method of identifying a compound useful in the IGF-independent modulation of angiogenesis comprising: (a) obtaining conditioned medium from dB-cAMP treated U87MG cell; (b) separating out components in the medium by conventional means; and (c) screening the separated components for IGF-independent modulation of angiogenesis or tumor growth.

### BRIEF DESCRIPTION OF THE DRAWINGS

FIG. 1. Effects of dB-cAMP on U87MG proliferation rates (A) and colony formation in a semi-solid agar (B). (A) U87MG were grown in the absence (open bars) or presence (closed bars) of 500 μM dB-cAMP for 6 days and their proliferation rates were determined using a CyQuant Proliferation Assay Kit as described in Materials and Methods. Each bar represents mean cell number per well±s.e.m. of 3 experiments run in quintuplicate. Asterisks indicate a significant (p<0.05, ANOVA followed by a Newman-Keuls multiple comparison test) difference between two treatments. (B&C) U87MG were grown in a semi-solid agar in the absence (B) or presence (C) of 500 μM dB-cAMP for 4 weeks and the number and size of colonies were evaluated as described in Materials and Methods. Calibration bar=500 μm.

FIG. 2. Representative photo-micrographs (magnification 40×) of capillary-like tubes formed by human brain endothelial cells (HBEC) grown in Matrigel™ and exposed to the following treatments: (A) serum-free D-MEM; (B) conditioned media of U87MG cells; C) conditioned media of dB-cAMP-treated U87MG cells; (D) conditioned media of U87MG supplemented with 500 μM dB-cAMP; (E) conditioned media of U87MG cells (F) conditioned media of U87MG cells pre-treated with 1 μg/ml of neutralizing anti-VEGF antibody. Formation of capillary like tubes was evaluated as described in Materials and Methods. Calibration bar=500 μm (G) Quantitative assessment of the total length of capillary-like tube network and the number of nodes in repeated experiment using conditions described in A, B-E, C and F. Bars are means±s.e.m. of 3-5 experiments. * indicates significance (p<0.05, ANOVA followed by Newman-Keuls) between D-MEM and U87MG CM. ⁺⁺ indicates significance (p<0.05, ANOVA followed by Newman-Keuls) between U87MG CM conditions without or with different treatments (dB-cAMP and VEGF Ab).

FIG. 3. Changes in protein levels/activity of selected genes differentially expressed between untreated and dB-cAMP-treated U87MG cells. (A) PAI-1 expression in U87MG in the absence (−) or presence (+) of dB-cAMP (500 μM, 6-day treatment) determined by Western-blot analysis. (B) Plasminogen activator activity (PAA) in U87MG cells in the absence (empty bar) or presence (full bar) of dB-cAMP (3-day treatment). Levels of secreted SPARC (C) and IGFBP-4 (D) determined by ELISA in CM of U87MG cells grown in the absence (empty bars) or presence (full bars) of dB-cAMP (500 μM, 6-day treatment). Bars in histograms are means±s.e.m. of six replicates. Asterisks indicate significant (p<0.05, t-test) difference between control and dB-cAMP-treated cells.

FIG. 4. The effect of IGFBP-4 on U87MG-induced capillary like tube formation by HBEC grown in Matrigel™. 4×10⁴ HBEC cells/well were plated in Matrigel™-precoated wells and cultured in following conditions: (A) serum-free D-MEM; (B) conditioned media of U87MG cells (CM); (C) conditioned media of U87MG cells supplemented with 500 ng/ml of recombinant IGFBP-4; (D) conditioned media of dB-cAMP (500 μM, 6 days)-treated U87MG cells (dB-cAMP-CM); (E & F) conditioned media of dB-cAMP (500 μM, 6 days)-treated U87MG cells pre-incubated with 15 μg/ml (15 IGFBP4 Ab, E) or 30 μg/ml (30 IGFBP4 Ab, F) of anti-IGFBP-4 antibody for 30 min at 37° C. Phase-contrast microphotographs were taken 18 h after treatments at 40× magnification. Calibration bar=500 μm. (G) Quantitative assessment of the total length of capillary-like tube network and the number of branching points in repeated experiment using conditions described in A-F. Bars are means±s.e.m of 3-5 experiments. * Indicates significance (p<0.05, ANOVA followed by Newman-Keuls) between D-MEM and U87MG CM. ⁺⁺ Indicates significance (p<0.05, ANOVA followed by Newman-Keuls) between U87MG CM in the absence or presence of IGFBP-4. # Indicates significance (p<0.05, ANOVA followed by Newman-Keuls) between dB-cAMP-treated U87MG CM in the absence or presence of different concentrations of neutralizing anti-IGFBP-4 antibody.

FIG. 5. Effects of IGFBP-4 on growth factor-induced capillary like tube formation by HBEC grown in Matrigel™. Histograms represent total length (left panels) and number of branching nodes (right panels) of the capillary-like tube network. HBEC were exposed to D-MEM (white bars), 150 ng/ml IGF-1 (A), 20 ng/ml VEGF (B), 100 ng/ml P1GF (C), or 20 ng/ml bFGF (D) in the absence (black bars) or presence (hatched bars) of 500 ng/ml IGFBP-4. Bars are means±s.e.m. of 3-5 experiments. * indicates significance (p<0.05, ANOVA followed by Newman-Keuls) between D-MEM-treated and growth factor-treated HBEC; ⁺⁺ indicates significance (p<0.05, ANOVA followed by Newman-Keuls) between growth factor-treated HBEC in the absence and presence of IGFBP-4.

FIG. 6. The effect of IGFBP-4 on colony formation by tumor cells grown in semi-solid agar. U87MG cells (A-C) and Hela cells (D-F) were grown in semi-solid agar in the absence (A, E) or presence (B, F) of 500 ng/ml IGFBP-4 over 4 weeks as described in Materials and Methods. Calibration bar=500 μm. Histograms show the total covered area per field (C, G) and the number of colonies (D, H) formed by U87MG (C, D) and Hela (G, H) cells in the absence (empty bars) or presence (full bars) of 500 ng/ml IGFBP-4. Bars are means±s.e.m. of 36 images obtained from two experiments done in triplicates. Asterisks indicates significant (p<0.05, t-test) difference between control and IGFBP-4-treated cells.

FIG. 7. Effects of the full length IGFBP-4 protein, N-terminal (NBP-4)- and C-terminal (CBP-4) IGFBP-4 protein fragments on U87MG CM- and growth factor-induced capillary like tube formation by HBEC grown in Matrigel™. Histograms represent total length (left panels) and number of branching nodes (right panels) of the capillary-like tube network. HBEC were exposed to D-MEM, U87MG CM (A), 150 ng/ml IGF-1 (B), 20 ng/ml bFGF (C), or 20 ng/ml VEGF (D) in the absence or presence of 500 ng/ml of IGFBP-4, NBP-4 or CBP-4. Bars are means±s.e.m. of 3-5 experiments. * indicates significance (p<0.05, ANOVA followed by Newman-Keuls) between either U87MG CM or growth factor-treated HBEC in the absence and presence of IGFBP-4, NBP-4 and CBP-4.

FIG. 8. The effect of IGFBP-4, N-terminal-(NBP-4) and C-terminal-(CBP-4) IGFBP-4 protein fragments on colony formation by U87MG grown in semi-solid agar. U87MG cells were grown in semi-solid agar in the absence (A, E) or presence of 500 ng/ml either IGFBP-4 (B, E), or NBP-4 (C, E) or CBP-4 (D, E) over 4 weeks as described in Materials and Methods. Calibration bar=500 μm. Histograms show the total covered area per field (E) formed by U87MG cells in the absence or presence of 500 ng/ml of either IGFBP-4 or NBP-4 or CBP-4. Bars are means±s.e.m. of 36 images obtained from two experiments done in triplicates. Asterisks indicates significant (p<0.05, t-test) difference between control and IGFBP-4-, NBP-4-, and CBP-4-treated cells.

FIG. 9. Internalization of CBP-4 conjugated to Alexa fluor647 (AF647-CBP-4) into human brain endothelial cells. Microphotographs were obtained 90 min after endothelial cell exposure to AF647-CBP-4 using confocal microscopy as described in Material and Methods. Internalized AF647-CBP-4 appears associated with lysosome-like structures.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

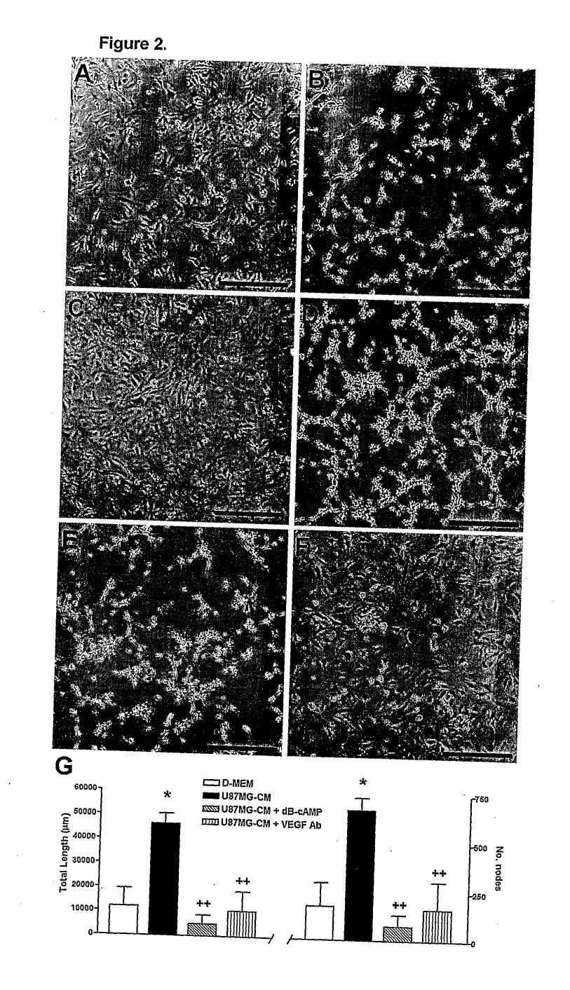

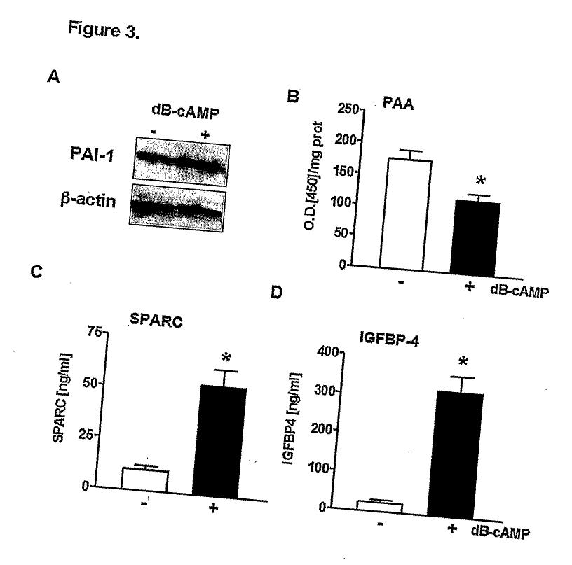

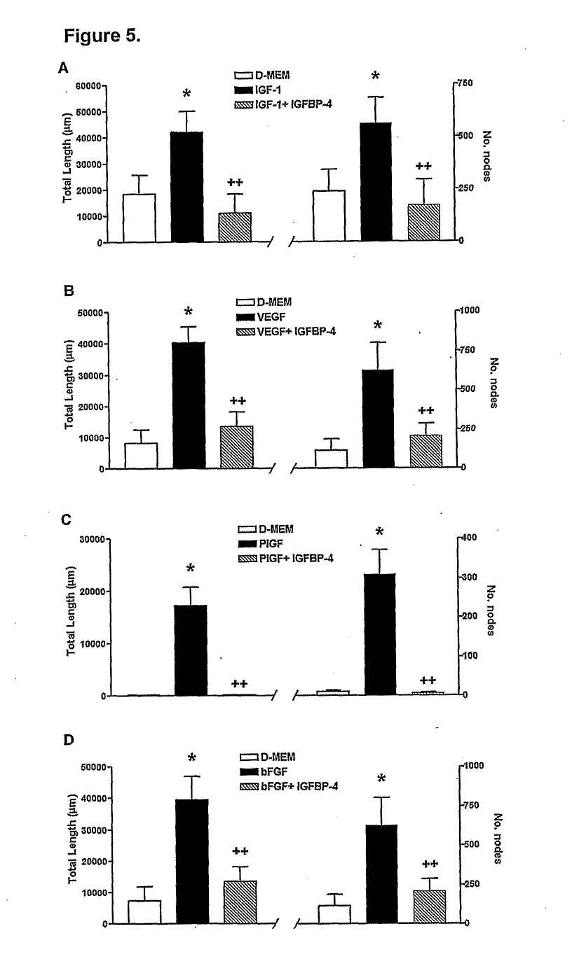

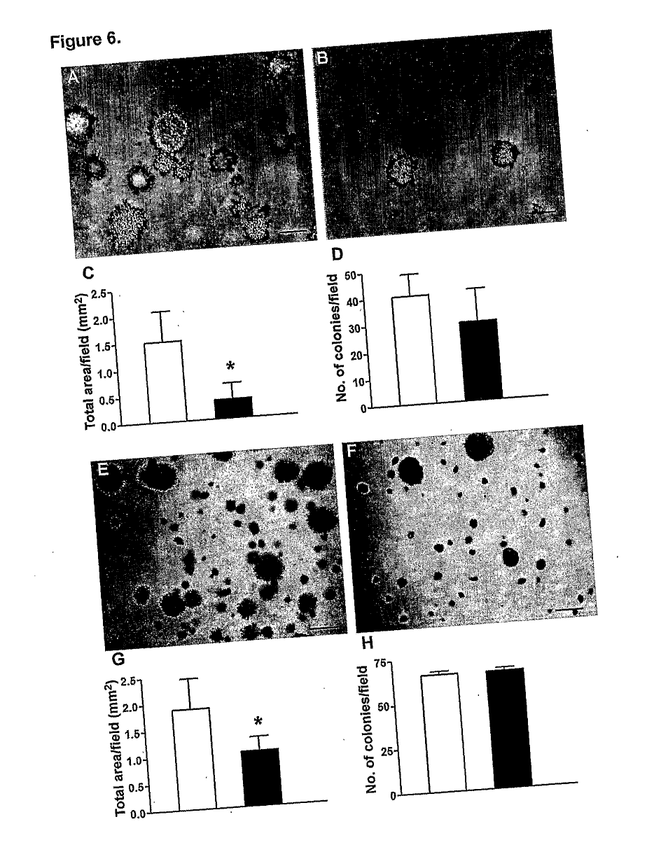

It is disclosed herein that dibutyryl cyclic AMP (dB-cAMP) blocks the angiogenic response of brain endothelial cells induced by glioblastoma cell (U87MG)-conditioned media (FIG. 2). A gene expression profiling approach used to identify downstream effectors responsible for dB-cAMP-mediated inhibition of U87MG-induced angiogenic properties led to the identification of potent and pleiotropic anti-angiogenic properties of the insulin-like growth factor binding protein (IGFBP)-4 secreted by db-cAMP-treated U87MG cells (Table 1 and IV, FIG. 3). IGFBP-4 antagonized angiogenic responses induced by U87MG and a variety of growth factors, including vascular endothelial growth factor-165 (VEGF₁₆₅), insulin-like growth factor (IGF)-1, placenta growth factor (P1GF), and basic fibroblast growth factor (bFGF) (FIG. 5). IGFBP4 also reduced U87MG (˜80%) and HELA (˜50%) colony formation in semi-solid agar (FIG. 6). Therefore, IGFBP-4 is a novel downstream effector of dB-cAMP with dual anti-angiogenic and anti-tumorigenic properties that may be used for suppressing tumor growth.

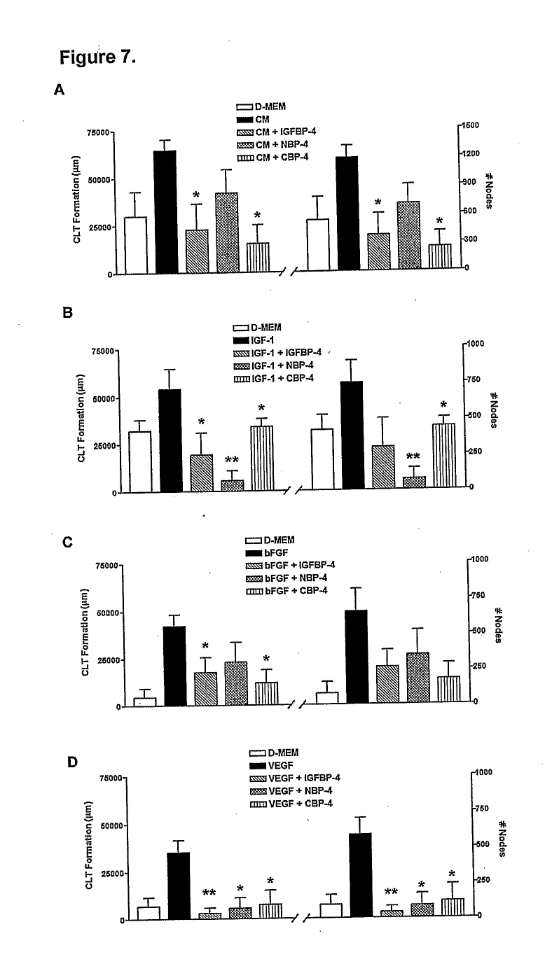

Studies designed to identify the IGFBP-4 protein domain(s) containing the anti-angiogenic activity revealed that the recombinant C-terminal (Table VI, SEQ ID No. 4, aa 155 to 258 of SEQ ID No. 1, numbering corresponding to the IGFBP-4 precursor, SWISS-PROT accession no. P22692) IGFBP-4 protein fragment was capable of completely blocking the angiogenic response induced by U87MG-conditioned media and a number of pro-angiogenic growth factors including IGF-1, bFGF, VEGF and P1GF in human brain endothelial cells (FIG. 7).

The recombinant N-terminal (Table V, SEQ ID No. 3, aa 1 to 156, numbering corresponding to the IGFBP-4 precursor (SEQ ID No. 1), SWISS-PROT accession no. P22692) IGFBP-4 protein fragment was able to abolish the angiogenic response induced by IGF-1 and VEGF (FIG. 7).

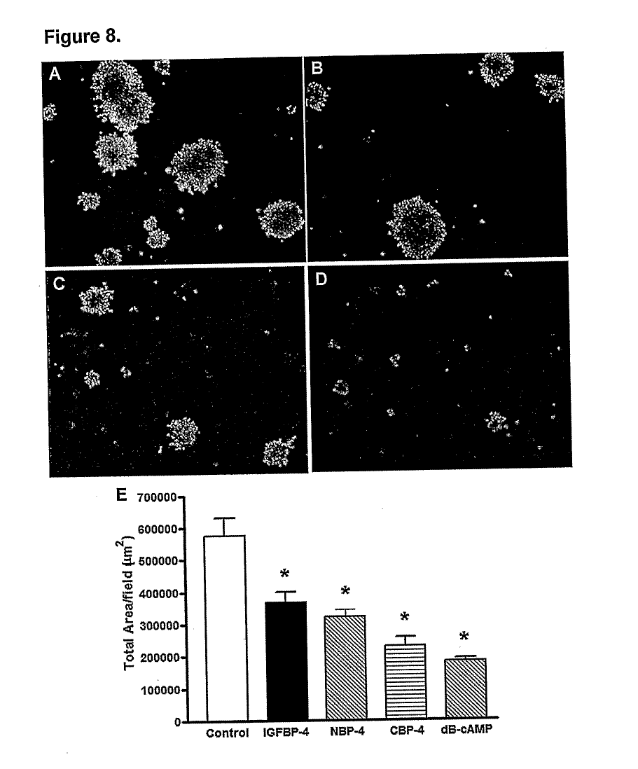

Studies of U87MG colony formation in soft-agar showed that both the C- and N-terminal IGFBP-4 fragments inhibited tumor growth (N-terminal: ˜50%, C-terminal: ˜55%) (FIG. 8).

The C-terminal IGFBP-4 fragment contains a thyroglobulin type-1 domain (Table VI, aa 200-249, numbering corresponding to the IGFBP-4 precursor, SWISS-PROT accession no. P22692) with the following consensus pattern [FYWHPVAS]-x(3)-C-x(3,4)-[SG]-x-[FYW]-x(3)-Q-x(5,12)-[FYW]-C-[VA]-x(3,4)-[SG]. Without restricting the invention to any particular mechanism or mode of action, it appears that the C-terminal IGFBP-4 fragment inhibits angiogenesis by inactivation of proteinase activities.

Tumor invasion, angiogenesis and metastasis are associated with altered lysosomal trafficking and increased expression of lysosomal proteases termed cathepsins. Several members of the cathepsins have been implicated in cancer progression. High expression levels of these cathepsins offer a reliable diagnostic marker for poor prognosis. Together with matrix metalloproteases and the plasminogen activator system, secreted cathepsins have been suggested to participate in the degradation of extracellular matrix, thereby enabling enhanced cellular motility, invasion and angiogenesis.

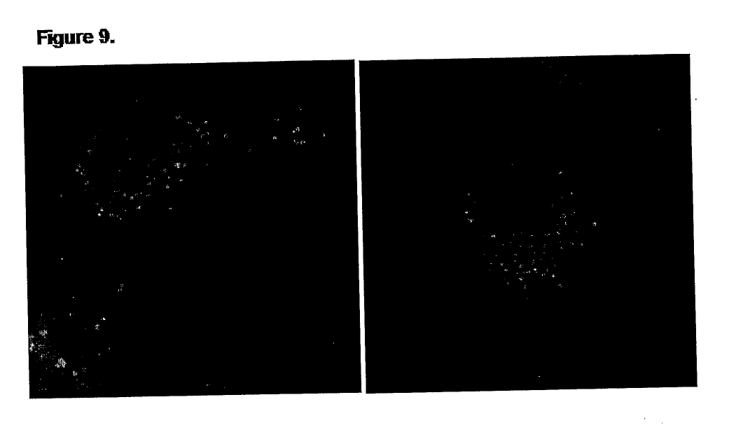

Confocal microscopy studies confirmed the ability of the C-terminal IGFBP-4 fragment conjugated to Alexa Fluor 647 to internalize into human brain endothelial cells and accumulate in lysosome-like structures (FIG. 9).

In an embodiment of the invention there is provided a composition comprising dB-cAMP-treated U87MG cells conditioned media.

In an embodiment of the invention there is provided a conditioned media composition from db-cAMP-treated U87MG cells with anti-angiogenic and anti-tumorigenic activity.

In an embodiment of the invention there is provided a method of identifying a compound useful in the IGF-independent modulation of angiogenesis comprising: (a) obtaining conditioned medium from dB-cAMP treated U87MG cell; (b) separating out components in the medium by conventional means (e.g. size, weight, charge by techniques such as column and/or thin layer chromatography or other suitable means) (c) screening the separated components for IGF-independent modulation of angiogenesis. In some cases the separated components can be further separated or purified.

For example, a number of genes up-regulated in dB-cAMP treated U87MG cells, are listed in Table 1. As will be appreciated by one of skill in the art, a number of fractionation schemes can easily be developed which can be used to isolate desired peptides or combinations of peptides based on their known biochemical properties, for example, charge, size, pI and the like. As such, identification of other anti-tumorigenic agents from the media can be done as described herein and is within the scope of the invention.

In an embodiment of the invention there is provided the use of dB-cAMP-treated U87MG cells conditioned media and/or components thereof derived from the treated cells in the inhibition of angiogenesis and suppression of tumor growth and/or the manufacture of a medicament useful for such purposes.

In an embodiment of the invention there is provided components of dB-cAMP-treated U87MG conditioned media, IGFBP-4, with potent anti-angiogenic and antitumorigenic properties.

In an embodiment of the invention there is provided a method of reducing angiogenesis by modulating the interaction of IGF with a receptor, comprising regulating the concentration of IGFBP-4 in the vicinity of the receptor.

In an embodiment of the invention there is provided an amino acid sequence useful in inhibiting angiogenic responses induced by a variety of growth factors in endothelial cells and/or invasive properties of glioblastoma cells. In some instances, the amino acid sequence is at least 70%, 75%, 80%, 85%, 90%, 95%, 98%, 99% or 100% identical in amino acid sequence to at least one of SEQ ID NO. 1, 2, 3, 4, 5, 6, 7 or 8. In, some instances, differences in amino acid sequence identity will be attributable to conservative substitutions wherein amino acids are replaced by amino acids having a similar size, charge and level of hydrophobicity.

In a preferred embodiment, the IGFBP-4 peptide comprises 20 or more consecutive amino acids of amino acids 200-249 of SEQ ID No. 1 or 20 or more consecutive amino acids of amino acids 155-258 of SEQ ID No. 1 or 20 or more consecutive amino acids of amino acids 1 to 258 of SEQ ID No. 1 or 20 or more or at least 20 consecutive amino acids of amino acids 1 to 155 of SEQ ID No. 1.

In a further aspect of the invention, the peptide comprises at least one amino acid sequence selected from the following:

(SEQ ID No. 6)

DEAIHCPPCSEEKLARGRPPVGCEELVREPGCGCCATCALGLGMPCGVYT
PRCGSGLRCYPPRGVEKPLHTLMHGQGVCMELAEIEAIQESLQPSDKDEG
DHPNNSFSPCSAHDRRCLQKHFAKIRDRSTSGGKMKVNGAPREDARPVPQ
GSCQSELHRAALERLAASQSRTHEDLYIIPIPNCDRNGNFHPKQCHPALD
GQRGKCWCVDRKTGVKLPGGLEPKGELDCHQLADSFRE;

(SEQ ID No. 7)

DEAIHCPPCSEEKLARCRPPVGCEELVREPGCGCCATCALGLGMPCGVYT
PRCGSGLRCYPPRGVEKPLHTLMHGQGVCMELAEIEAIQESLQPSDKDEG
DHPNNSFSPCSAHDRRCLQKHFAKIRDRSTSGGKM;

(SEQ ID No. 8)

KVNGAPREDARPVPQGSCQSELHRALERLAASQSRTHEDLYIIPIPNCDR
NGNFHPKQCRPALDGQRGKCWCVDRKTGVKLPGGLEPKGELDCHQLADSF
RE;

(SEQ ID No. 9)

DEAIHCPPCSEEKLARCRPP;

(SEQ ID No. 10)

EEKLARCRPPVGCEELVREP;

(SEQ ID No. 11)

PVGCEELVREPGCGCCATCA;

(SEQ ID No. 12)

PGCGCCATCALGLGMPCGVY;

(SEQ ID No. 13)

ALGLGMPCGVYTPRCGSGLR;

(SEQ ID No. 14)

YTPRCGSGLRCYPPRGVEKP;

(SEQ ID No. 15)

RCYPPRGVEKPLHTLMHGQG;

(SEQ ID No. 16)

PLHTLMHGQGVCMELAEIEA;

(SEQ ID No. 17)

VCMELAEIEAIQESLQPSDK;

(SEQ ID No. 18)

AIQESLQPSDKDEGDHPNNS;

(SEQ ID No. 19)

KDEGDHPNNSFSPCSAHDRR;

(SEQ ID No. 20)

SFSPCSAHDRRCLQKHFAKI;

(SEQ ID No. 21)

RCLQKHFAKIRDRSTSGGKM;

(SEQ ID No. 22)

IRDRSTSGGKMKVNGAPRED;

(SEQ ID No. 23)

MKVNGAPREDARPVPQGSCQ;

(SEQ ID No. 24)

ARPVPQGSCQSELHRALERL;

(SEQ ID No. 25)

QSELHRALERLAASQSRTHE;

(SEQ ID No. 26)

LAASQSRTHEDLYIIPIPNC;

(SEQ ID No. 27)

EDLYIIPIPNCDRNGNFHPK;

(SEQ ID No. 28)

CDRNGNFHPKQCHPALDGQR;

(SEQ ID No. 29)

QCHPALDGQRGKCWCVDRKT;

(SEQ ID No. 30)

RGKCWCVDRKTGVKLPGGLE;

(SEQ ID No. 31)

RKTGVKLPGGLEPKGELDCH;

(SEQ ID No. 32)

EPKGELDCHQLADSFRE;

(SEQ ID No. 33)

KVNGAPREDARPVPQGSCQSELHRALERLAASQSRTHEDLYIIPIPNCDR
N;

(SEQ ID No. 34)

GNFHPKQCHPALDGQRGKCWCVDRKTGVKLPGGLEPKGELDCHQLADSFR
E;

(SEQ ID No. 35)

KVNGAPREDARPVPQGS;

(SEQ ID No. 36)

CQSELHRALERLAASQS;

(SEQ ID No. 37)

RTHEDLYIIPIPNCDRN;

(SEQ ID No. 38)

GNFHPKQCHPALDGQRG;

(SEQ ID No. 39)

KCWCVDRKTGVKLPGGL;

(SEQ ID No. 40)

EPKGELDCHQLADSFRE;

(SEQ ID No. 41)

GAPREDARPVPQGSCQSELH;

(SEQ ID No. 42)

REDARPVPQGSCQSELHRAL;

(SEQ ID No. 43)

RPVPQGSCQSELHRALERLA;

(SEQ ID No. 44)

PQGSCQSELHRALERLAASQ;

(SEQ ID No. 45)

SCQSELHRALERLAASQSRT;

(SEQ ID No. 46)

SELHRALERLAASQSRTHEDL;

(SEQ ID No. 47)

HRALERLAASQSRTHEDLYII;

(SEQ ID No. 48)

LERLAASQSRTHEDLYIIPIP;

(SEQ ID No. 46)

LAASQSRTHEDLYIIPIPNCD;

(SEQ ID No. 50)

SQSRTHEDLYIIPIPNCDRNG;

(SEQ ID No. 51)

RTHEDLYIIPIPNCDRNGNFH;

(SEQ ID No. 52)

EDLYIIPIPNCDRNGNFHPKQ;

(SEQ ID No. 53)

YIIPIPNCDRNGNFHKQCHP;

(SEQ ID No. 54)

PIPNCDRNGNFHPKQCHPALD;

(SEQ ID No. 55)

NCDRNGNFHPKQCHPALDGQR;

(SEQ ID No. 56)

RNGNFHPKQCHPALDGQRGKC;

(SEQ ID No. 57)

NFHPKQCHPALDGQRGKCWCV;

(SEQ ID No. 58)

PKQCHPALDGQRGKCWCVDRK;

(SEQ ID No. 59)

CHPALDGQRGKCWCVDRKTGV;

(SEQ ID No. 60)

ALDGQRGKCWCVDRKTGVKLP;

(SEQ ID No. 61)

GQRGKCWCVDRKTGVKLPGGL;

(SEQ ID No. 62)

GKCWCVDRKTGVKLPGGLEPK;

(SEQ ID No. 63)

CWCVDRKTGVKLPGGLEPKGE;

(SEQ ID No. 64)

DRKTGVKLPGGLEPKGELDCH;

(SEQ ID No. 65)

TGVKLPGGLEPKGELDCHQLA;

(SEQ ID No. 66)

KLPGGLEPKGELDCHQLADSF;
and

(SEQ ID No. 67)

PGGLEPKGELDCHQLADSFRE

In a further embodiment, the peptide comprises an amino acid sequence that is at least 70%, 75%, 80%, 85%, 90%, 95%, 98%, 99% or 100% identical to amino acids 200-249 of SEQ ID No. 1 or amino acids 155-258 of SEQ ID No. 1. As will be appreciated by one of skill in the art, suitable substitutions may be determined by comparing the IGFBP-4 sequence with other IGFBP family members and/or other thyroglobulin domains known in the art. Specifically, amino acid locations within IGFBP-4 likely to tolerate substitution are not likely to be highly conserved between IGFBP family members or between thyroglobulin domains, as shown in Table 7. Furthermore, tolerated conserved substitutions may be determined by comparing the sequences as well. It is of note that pairwise alignment of IGFBP-4 with the rest of the IGFBP members indicates that the percent of homology of these sequences varies between 54-70%.

In other embodiments, the IGFBP-4 peptide sequence may be flanked on either side or both by additional amino acids which may or may not be ‘native’ IGFBP-4 sequence or may be within a carrier or presenting peptide as known in the art.

In an aspect of the invention there are provided nucleic acid sequences encoding one or more of the amino acid sequences described above.

In an embodiment of the invention there is provided the use of IGFBP-4 or a fragment thereof, where the fragment is or comprises the C-terminal (SEQ ID No. 8) IGFBP-4 protein fragment or the thyroglobulin domain (SEQ ID No 5) located in the C-terminal region of the IGFBP-4 protein or the N-terminal region of the IGFBP-4 protein (SEQ ID No. 7), or a peptide that comprises an amino acid sequence that is at least 70%, 75%, 80%, 85%, 90%, 95%, 98%, 99% or 100% identical to one of SEQ ID No. 5, SEQ ID No. 7 or SEQ ID No. 8 to inhibit angiogenesis or modulate angiogenic responses.

Angiogenesis is the formation of new blood vessels from pre-existing capillaries. There are different methods to evaluate angiogenesis in vitro and in vivo. The method used in our studies consists in seeding human brain microvascular endothelial cells on Matrigel, which is an active matrix material resembling the mammalian cellular basement membrane. Endothelial cells seeded on Matrigel behave as they do in vivo and when submitted to an angiogenic stimuli reorganize forming a complex network of capillary-like tubes. The total length of the capillary-like tube network as well as the number of branching point (nodes) formed by the endothelial cells directly correlate with the potency of the angiogenic stimuli.

Thus, as will be apparent to one of skill in the art, there are many ways of determining angiogenesis or more precisely determining an increase or decrease in angiogenesis compared to a control and that such methods are within the scope of the invention.

In an embodiment of the invention there is provided the use of IGFBP-4 or a fragment or variant thereof, where the fragment is preferentially a C-terminal IGFBP-4 protein fragment or the thyroglobulin domain located in the C-terminal region of the IGFBP-4 protein, to inhibit protease activity. For example, SEQ. ID. NO. 4 or SEQ. ID. NO. 5 or SEQ ID No. 7 may be used in certain instances. In a preferred embodiment, the IGFBP-4 fragment is an active fragment or a biologically active fragment, that is, a protease inhibitory fragment.

In an embodiment of the invention there is provided the ability of the C-terminal (SEQ ID No. 4) IGFBP-4 protein fragment and smaller peptides of this region to internalize in target cells

In an embodiment of the invention there is provided the use of IGFBP-4 or a fragment or a fragment and/or variant thereof, to inhibit tumor growth in mammal.

In an embodiment of the invention there is provided the use of an amino acid sequence having at least 70% sequence identity to SEQ. ID. NO. 3, 4, 5, 6, 7 or 8 to inhibit tumor growth in a mammal. In some cases sequence identity is preferably at least 75%, 80%, 85%, 90%, 95%, 97%, 98%, 99% or 100%. In some cases the sequence includes non-natural and/or chemically modified amino acids.

In an embodiment of the invention there is provided use of IGFBP-4 or a fragment or variant thereof as described above in modulating the activity of or biological response to one or more growth factors. In some cases the growth factor whose biological activity is modulated is at least one of: IGF-I, VEGF₁₆₅, P1GF and bFGF.

In an embodiment of the invention there is provided a method of inhibiting angiogenic transformation of endothelial cells comprising administering IGFBP-4 or a fragment or variant thereof as described above. As discussed above, there are many methods known in the art for measurement of angiogenesis. In some embodiments, inhibition of angiogenesis may be based on a comparison between a treatment group which is administered an effective amount of the IGFBP-4 fragment as described herein and an untreated or mock-treated control. It is of note that the control would not necessarily need to be repeated each time.

In an embodiment of the invention there is provided a method of decreasing angiogenesis in a mammalian subject in need of such treatment, comprising administering IGFBP-4 or a fragment or variant thereof.

In an embodiment of the invention there is provided a method of decreasing tumor growth or decreasing metastasis in a mammalian subject, comprising administering IGFBP-4 or a fragment or variant thereof to a subject in need of such treatment. As discussed above, there are many methods known in the art for measurement of tumor growth and metastasis. In some embodiments, inhibition of tumor growth or metastasis may be based on a comparison between a treatment group which is administered an effective amount of the IGFBP-4 fragment as described herein and an untreated or mock-treated control. It is of note that the control would not necessarily need to be repeated each time.

In some embodiments, the IGFBP-4 peptide as discussed herein may be combined with a matrix, gel or other similar compound such that the IGFBP-4 peptide is substantially retained in a localized area following application thereof to the site of interest.

In an embodiment of the invention there is provided the use of SEQ ID NO 1, 2, 3, 4, 5, 6, 7 or 8 or a variant or fragment thereof in the manufacture of a medicament useful for the reduction of angiogenesis or tumor growth in a mammal. In some instances, the amino acid sequences of the invention will be labeled with radioactive isotopes or fluorescent tags for detection or conjugated to hydrophobic sequences to increase their permeability through biologic membranes.

In some instances, the amino acid sequences of the invention will include non-natural amino acids and/or modified amino acids. Modifications of interest include cyclization, derivitivization and/or glycosylation of one or more functional groups.

In an embodiment of the invention there is provided the use of expression vectors (e.g. bacterial, viral, mammalian, yeast, etc) for generating recombinant protein of one or more of the amino acid sequences described above.

In an embodiment of the invention there is provided the use of viral vectors (e.g. retrovirus, adenovirus, adeno-associated virus, herpes-simplex) or non-viral methods of DNA transfer (e.g. naked DNA, liposomes and molecular conjugates, nanoparticles) for delivery and expression of one or more of the amino acid sequences described above in mammalian organs to inhibit pathological angiogenesis or tumor growth.

In an embodiment of the invention there is provided a composition useful in the treatment of mammals with tumors, comprising IGFBP-4 or a fragment or variant thereof, and a pharmaceutically acceptable carrier. In some instances the composition will be in dosage form. In some instances the carrier will be selected to permit administration by injection. In some cases the carrier will be selected to permit administration by ingestion. In some cases the carrier will be selected to permit administration by implantation. In some cases the carrier will be selected to permit transdermal administration.

In an embodiment of the invention there is provided a composition comprising IGFBP-4 or a fragment or variant thereof together with a least one additional modulator of angiogenesis and a suitable carrier.

It is of note that additional modulators are known in the art.

As used herein, an ‘effective amount’ of an IGFBP-4 peptide refers to an amount that is sufficient to accomplish at least one of the following: reduction of angiogenic transformation; inhibition of angiogenic transformation; reduction of angiogenesis; inhibition of angiogenesis; reduction of rate of tumor growth; inhibition of tumor growth; reduction of tumor size; inhibition of metastasis and reduction of metastatic frequency. As will be appreciated by one of skill in the art, the exact amount may vary according to the purification and preparation of the medicament as well as the age, weight and condition of the subject.

In an embodiment of the invention there is provided the use of a polypeptide sequence comprising at least one thyroglobulin type-1 domain in modulating angiogenesis in a mammal.

In an embodiment of the invention there is provided the use of a polypeptide sequence comprising the consensus pattern [FYWHPVAS]-x(3)-C-x(3,4)-[SG]-x-[FYW]-x(3)-Q-x(5,12)-[FYW]-C-[VA]-x(3,4)-[SG] in modulating angiogenesis in a mammal. In some cases this sequence will be present in 2, 3, 4, 5, 6, or more copies.

In an embodiment of the invention there is provided use of a polypeptide sequence comprising at least one contiguous amino acid sequence [FYWHPVAS]-x(3)-C-x(3,4)-[SG]-x-[FYW]-x(3)-Q-x(5,12)-[FYW]-C-[VA]-x(3,4)-[SG] and having at least 70% sequence identity to SEQ. ID. NO. 4 to inhibit protease activity. In some cases 75%, 80%, 85%, 90%, 95%, 98%, 99% or 100% sequence identity will be desirable. In some cases the polypeptide sequence will not be as long as SEQ. ID. NO. 4 but will have the specific contiguous sequence and the desired level of sequence identity with respect to its actual length. In some cases the polypeptide sequence will include at least one non-natural and/or chemically modified amino acid.

In an embodiment of the invention there is provided the use of a polypeptide sequence comprising at least one contiguous amino acid sequence selected from the group consisting essentially of: PNC, QC, and CWCV in modulating angiogenesis in a mammal. In some cases at least two such sequences will be present. In some instances all three sequences will be present. In some instances one or more sequences will be present in more than one copy. In some instances the polypeptide sequence will also have, along the balance of its length, at least 70%, 75%, 80%, 85%, 90%, 95%, 98%, 99% or 100% identical in sequence to the corresponding portion of SEQ. ID. NO. 4.

In an embodiment of the invention there is provided amino acid sequences and the use thereof in modulating angiogenesis and/or protease activity. Sequences of interest include: A₁ A₂ PNC A₆ A₇ A₈ G A₁₀ A₁₁ A₁₂ A₁₃ A₁₄ QC A₁₇ A₁₈ A₁₉ A₂₀ A₂₁ A₂₂ A₂₃ A₂₄ G A₂₆ CWCV A₃₁ A₃₂ A₃₃ A₃₄ G A₃₆ A₃₇ A₃₈ A₃₉ G A₄₁ A₄₂ A₄₃ A₄₄ A₄₅ A₄₆ A₄₇ A₄₈ A₄₉ A₅₀ C. In some instances, amino acids designated “A” can be any natural or unnatural amino acid, including chemically or biologically modified amino acids. In some instances, one or more of the amino acids designated “A” will be selected from one of the corresponding amino acids occurring at the corresponding location on one or more of the IGFBF sequences, including those shown in Table VII. In some instances one or both of A₃₂ and A₄₇ may not be present.

In an embodiment of the invention there is provided use of a protease inhibitor in modulating angiogenesis in a mammal. In some instances, the protease inhibitor is an inhibitor of at least one of a cysteine protease.

In an embodiment of the invention there is provided a composition comprising a cysteine protease inhibitor and a pharmaceutically acceptable carrier. In some instances such a composition may be used in modulating angiogenesis and/or tumour growth in a mammal.

In an embodiment of the invention there is provided use of a protease inhibitor in the manufacture of a medicament useful in the modulation of angiogenesis and/or tumour development in a mammal.

It will be understood that, while possible mechanisms of action may be discussed, the invention is not limited to any particular mechanism or mode of action.

### Materials and Methods

### Cell Cultures

The human glioma cell line U87MG was established from surgically removed type III glioma/glioblastoma and obtained from ATCC. The human cervical epithelial adenocarcinoma cell line, Hela, was kindly provided by Dr. Maria Jaramillo (Biotechnology Research Group, National Research Council Canada, Montreal, Canada). Cells (5×10⁴ cells/ml) were plated in poly-L-lysine pre-coated dishes and grown at 37° C. in D-MEM supplemented with 100 U/mi penicillin, 100 μg/ml streptomycin and 10% heat-inactivated fetal bovine serum (FBS) (HyClone, Logan, Utah) in humidified atmosphere of 5% CO₂/95% air. 500 μM dB-cAMP was added to the media for 3 days and replaced with serum-free D-MEM containing 500 μM dB-cAMP for 3 additional days. Control cells were subjected to the same protocol without dB-cAMP addition. Conditioned media of both control and dB-cAMP treated cells were collected and filtered (Millex-GV sterilizing filter membrane, 0.22 μm). Cells were then harvested for molecular and biochemical assays.

Human brain endothelial cells (HBEC) were obtained from small intracortical microvessels and capillaries (20-112 μm) harvested from temporal cortex from patients treated surgically for idiopathic epilepsy. Tissues were obtained with approval from the Institutional Research Ethics Committee. HBEC were separated from smooth muscle cells with cloning rings and grown at 37° C. in media containing Earle's salts, 25 mM 4-(2-hydroxyethyl)-1-piperazineethanesulfonic acid (HEPES), 4.35 g/L sodium bicarbonate, and 3 mM L-glutamine, 10% FBS, 5% human serum, 20% of media conditioned by murine melanoma cells (mouse melanoma, Cloudman S91, clone M-3, melanin-producing cells), 5 μg/ml insulin, 5 μg/ml transferrin, 5 ng/ml selenium, and 10 μg/ml endothelial cell growth supplement (Stanimirovic et al., 1996). HBEC cultures were routinely characterized morphologically and biochemically. More than 95% of cells in culture stained immunopositive for the selective endothelial markers, angiotensin II-converting enzyme and Factor VIII-related antigen, incorporated fluorescently labelled Ac-LDL, and exhibited high activities of the blood-brain barrier-specific enzymes, γ-glutamyltranspeptidase and alkaline phosphatase (Stanimirovic et al., 1996).

### Proliferation Assay

Proliferation rates of U87MG cells were determined using CyQUANT® Cell Proliferation Assay Kit (Molecular Probes, Inc., Eugene, Oreg.). Briefly, 3000 cells were plated in 96-well microplates in 150 μl of either D-MEM/1% FBS alone or supplemented with 500 μM dB-cAMP for 6 days. Cells were fed every two days and harvested at days 2, 3, 5 and 6 by washing with HBSS, blotting microplates dry and storing at −80° C. until analysis. For cell density determination, plates were thawed at room temperature, 200 μl of CyQUANT GR dye/lysis buffer was added to each well and plates were incubated in the dark for 5 min. Sample fluorescence was measured (485 nm ex/530 nm em) in a cytofluorimeter plate reader (Bio-Tek FL600) and fluorescence values converted into cell numbers from cell reference standard curves.

### Growth in Semi-Solid Agar

Anchorage-independent growth of U87MG and Hela cells in the absence or presence of either dB-cAMP, IGFBP-4, NBP-4 or CBP-4 was examined in semi-solid agar. D-MEM containing 10% FBS was warmed to 48° C. and diluted with Bacto-Agar to make a 0.6% (w/v) agar solution; 3 ml of agar solution was poured into 60 mm plates. 2 ml of 0.6% agar solution containing 25,000 cells±treatment (either 500 μM dB-cAMP or 500 ng/ml IGFBP-4) was then poured over the solidified bottom agar layer. The solidified cell layer was covered with 500 μl D-MEM±treatment which was replaced every three days over a 20-25 day period. Number and size of colonies formed were analyzed under the microscope (Olympus 1×50). Phase contrast images (6 fields/dish) were captured using a digital video camera (Olympus U-CMT) and analyzed with Northern Eclipse v.5.0 software. Each experiment was done in triplicate. Capillary-like tube (CLT) formation

In vitro angiogenesis was assessed by endothelial tube formation in growth factor reduced Matrigel™ (BD Bioscience, Bedford, Mass.). 24-well plates were coated with 300 μl of unpolymerized Matrigel™ (5-7 mg prot/ml) and allowed to polymerize for 90 min at 37° C. HBEC (40,000 cells) were suspended in 500 μl D-MEM alone, D-MEM containing growth factors (150 ng/ml IGF-I, 20 ng/ml VEGF₁₆₅, 100 ng/ml P1GF, or 20 ng/ml bFGF—R&D Systems, Inc., MN, USA), or serum-free CM (collected as described in Cell Cultures) from U87MG cells grown in the absence or presence of dB-cAMP, and then plated into Matrigel™-coated wells. In a set of experiments, 500 ng/ml of either full length recombinant IGFBP-4, NBP-4 or CBP-4 were co-applied with growth factors (IGF-I, VEGF₁₆₅, P1GF, or bFGF) or U87MG CM. In other experiments, CM from dB-cAMP-treated or untreated U87MG cells were respectively pre-incubated with 15-30 μg/ml of anti-IGFBP-4 antibody (Sigma, MO, USA) or 1 μg/ml of polyclonal anti-VEGF antibody (R&D systems, Inc) at 37° C. for 30 min and then mixed with HBEC. CLT formation was analyzed after 24 h using an Olympus 1×50 microscope. Phase contrast images were captured with a digital video camera (Olympus U-CMT) and analyzed using Northern Eclipse v.5.0 software. Microphotographs were thresholded, converted to binary images and skeletonized. The total length of the CLT networks and the number of nodes (branching points) formed by HBEC in the center of the well (˜80% of the total surface) were quantified. Experiments were performed in duplicate wells and repeated three times, using 3-5 different HBEC isolations.

### Microarray Experiments

Total RNA from U87MG cells incubated in the absence or presence of dB-cAMP was isolated using Trizol reagent (Gibco BRL, Gaithersburg, Md.) and further purified by RNeasy kit (Qiagen, Mississauga, Canada) according to manufacturer's protocol.

Differential gene expression between non-treated and dB-cAMP-treated U87MG cell was studied using 19.2K human cDNA microarrays from the University Health Network (UHN) Microarray Centre. Detailed information about the genes and expressed sequence tags (EST's) spotted on the slides is available at http://www.microarrays.ca/support/glists.html. Briefly, 20 μg RNA from each experimental treatment was primed with 1.5 μl AncT mRNA primer (5′-T₂₀VN, 100 pmoles/μl) in the presence of 1 μl of either Cy3- or Cy5-dCTP (Amersham Biosciences, Quebec, Canada), 3 μl of 20 mM dNTP (-dCTP), 1 μl of 2 mM dCTP, 4 μl of 0.1 M dithiothreitol (DTT), 5 ng Arabadopsis chlorophyl synthetase gene (positive control) and 8 μl 5×First Strand reaction buffer (Invitrogen Life Technologies, ON, Canada) in a final volume of 40 μl. The mixture was incubated in the dark at 65° C. for 5 minutes and then at 42° C. for 5 minutes. RNA was then reversed transcribed with 2 μl Superscript II reverse transcriptase enzyme (Invitrogen Life Technologies) at 42° C. for 3 h. The RNA was hydrolyzed with 4 μl of 50 mM EDTA (pH 8.0) and 2 μl of 10M NaOH at 65° C. for 20 min. Samples were neutralized with 1.5 μl of 5M acetic acid. The two probes (one labeled with Cy3 and the other with Cy5) were mixed and the cDNA precipitated with 100 μl isopropanol on ice for 60 min; samples were spun for 10 minutes at 4° C. and isopropanol was removed. cDNA was rinsed with ice-cold 70% ethanol, pelleted again and resuspended in 5 μl distilled water.

The fluorescent probes were mixed with 80 μl of DIG Easy Hyb solution (Roche, Mississauga, Canada), 1.6 μl of 25 mg/ml yeast tRNA (Invitrogen Life Technologies) and 4 μl of 10 mg/ml salmon sperm DNA (Sigma, MO, USA), heated at 65° C. for 2 minutes and then cooled to room temperature. Slides were covered with 85 μl of hybridization mixture and incubated at 37° C. overnight. Slides were then washed 3 times with pre-warmed 1×SSC 0.1% SDS, and rinsed with 1 X SSC and spin dried.

cDNA microarrays were scanned at 535 nm (Cy3) and 635 nm (Cy5) using dual-color confocal laser scanner ScanArray 5000 (GSI Lumonics, Billerica, Mass., USA). Images were analyzed using QuantArray® Micorarray Analysis Software v.2.0 (GSI Lumonics). Relative cDNA expression levels were quantified by comparing fluorescent signals obtained from Cy3- and Cy5-labeled probes.

For statistical purposes, 4 microarray replicates (dye-flip) were performed. Using an in-house custom-developed software (Normalizer™), the background of each spot was evaluated by counting pixel intensities in an area surrounding the spot and the subarray median background was subtracted from the fluorescent value of each spot. The log₂ raw net signals from each subarray channel (Cy3 or Cy4) were normalized using a linear regression algorithm. Spots showing low fluorescent intensity (below 5% of the range of intensities for each dye), high fluorescent intensity (above the 98% of the range of intensities for each dye), and/or high duplicate variation (ratio difference of duplicate spots representing the same EST 1.5-fold greater than the threshold) were removed from the data set. Since each of 19.2 K ESTs represented on the microarray slide was arrayed in duplicate, average of the fluorescent ratio for each duplicate spot was calculated and t-test analysis was applied to the 4 data sets. Significant differential expression was accepted when the normalized mean intensity ratio was >1.5-fold and probability scores lower than 0.05 using t-test analysis (Table I and II).

### Real-Time PCR

Total RNA was isolated from cells with TRIzol Reagent (Gibco BRL). The RNA (1 μg) was primed with oligo (dT)₁₂₋₁₈ primers (0.5 μg/μg RNA, Gibco BRL) and reverse transcribed with 1-3 U of avian myeloblastosis virus reverse transcriptase (AMV RT, Promega) in a final volume of 20 μl. Completed RT reactions were diluted to 40 μl with water. Control reactions without the enzyme were run in parallel to monitor for potential genomic contamination. Primers were designed (Primer Express Software v2.0) for genes of interest (Table III) using Primer Express 2.0 program. Real-Time PCR was carried out with SYBR Green PCR Core Reagents Kit (Applied Biosystems, CA, USA) using the GeneAmp® 5700 Sequence Detection System (Perkin Elmer Applied Biosystems). A cDNA pool serially diluted from 1:10 to 1:1000 was used to generate standard curves. Reactions were performed in 20 μl reaction mixture containing 1×SYBR PCR buffer (Perkin-Elmer), 200 μM of each dATP, dCTP, dGTP and 400 μM dUTP, 0.025 U/μl AmpliTaq Gold, 0.01 U/μl AmpEraseUNG (uracil-N-glycosylase), 3 mM MgCl₂, 120 nM of each primer and 2 μl of cDNA. The PCR mixture was first incubated at 50° C. for 2 min to activate AmpErase UNG and prevent the re-amplification of carryover PCR products, and then at 95° C. for 10 min for AmpliTaq Gold polymerase activation. The thermal PCR conditions were 10 sec denaturation at 95° C. and 1 min annealing-extension at 60° C. for 40 cycles. Fluorescence was detected at the end of every 60° C. phase. To exclude the contamination of unspecific PCR products such as primer dimers, melting curve analysis was performed for all final PCR products after the cycling protocol.

The PCR cycle number at which fluorescence reaches a threshold value of 10 times the standard deviation of baseline emission was used for quantitative measurements. This cycle number represents the cycle threshold (Ct) and is inversely proportional to the starting amount of target cDNA. The relative amount of the gene of interest was extrapolated from the corresponding standard curve. The data was normalized to the housekeeping gene β-actin (ACTB).

Representative PCR products were purified and subjected to automatic fluorescence sequencing. BLAST program was used to estimate the percent of identity of the PCR sequences with the corresponding fragments of the published cloned human genes.

### Western-Blot

Cellular proteins were extracted using CHAPS buffer. Proteins were separated on a 10% SDS-PAGE gel and transferred onto nitrocellulose membranes. Membranes were blocked with 5% instant skim dry milk in PBST for 1 hour, then washed twice for 5 min with PBST. Blots were probed with 1:500 dilution of the PAI-1 primary antibody (Biogenesis Ltd, England, UK) in 2% skim milk in PBST supplemented with 10 mM sodium azide overnight at 4° C. After washing in PBST, membranes were incubated with HRP-labeled anti-mouse IgG secondary antibody (NEN Life Science Products, USA; 1:5000) for 1 h at room temperature. Bands were visualized using Western Blot Chemiluminescence Reagent Plus kit (NEN™ Life Science Products) and the Fluor-S™ multiImager (BioRad Lab., Hercules, Calif., USA).

### ELISA

Levels of secreted VEGF, P1GF, bFGF, SPARC, IGFBP-4 and IGF-I in serum-free CM (described in Cell cultures) from U87MG cells were determined using colorimetric “sandwich” ELISA kits (R&D Systems Inc.), respectively, according to manufacture's protocols. Each sample was run in duplicate; three independent experiments were performed.

### Plasminogen Activator Activity Assay

Plasminogen activator activity (PAA) was determined by a spectrophotometric method using the chromogenic substrate S-2251 (D-Val-Leu-Lys p-nitroanilide dihydrochloride). Cells were plated on poly-L-lysine pre-coated 96-well plates and grown for three days in 100 μl media containing either D-MEM/10% FBS alone or supplemented with 500 μM dB-cAMP. Cells were washed 3 times and incubated for 2 h at 37° C. in phenol red-free D-MEM containing 2 c.u./ml plasminogen (DiaPharma Group, Ohio, USA) and 2 mM chromogenic substrate S-2251 (DiaPharma Group, Ohio, USA). The cleavage of 4-nitroaniline from the substrate by plasminogen activator was measured photometrically at 405 nm. Protein levels were measured with BioRad protein assay and PPA was expressed as a function of protein content in cell extracts.

### Production of Recombinant Full-Length IGFBP-4 and C- and N-terminal IGFBP-4 Protein Fragments.

Full length IGFBP4 (Accession number BCO16041; MGC:20162) was amplified with forward (F1: 5′-TAAGAATTCGCCACCATGCTGCCCCTCTGCCT-3′, SEQ ID No. 68) and reverse (R1: 5′-TTAGGATCCACCTCTCGAAAGCTGTCAGCC-3′, SEQ ID No. 69) primers, digested with EcoRI and BamHI and cloned in-frame into pTT5SH8Q1 expression plasmid containing the C-terminal Steptag-II/(His)₈GGQ dual tags (a smaller version of pTTSH8Q1 vector. IGFBP4 N-terminal domain (nt 1-156) was amplified with forward (F1) and reverse (R2: 5′-TTAGGATCCATCTTGCCCCCACTGGT-3′, SEQ ID No. 70) primers, digested and cloned as for the full-length. The IGFBP4 C-terminal domain (nt 155-258) was amplified with forward (F2: 5′-GCCGCTAGCAAGGTCAATGGGGCGCCCCGGGA-3′, SEQ ID No. 71) and reverse (R1) primers, digested with NheI and BamHI and ligated in-frame into pYD1 plasmid (pTT5SH8Q1 vector with SEAP signal peptide MLLLLLLLGLRLQLSLGIA, SEQ ID No. 72). Cells were transfected with PEI essentially as described with the following modifications: 293-6E cells (293-EBNA1 clone 6E) growing as suspension cultures in Freestyle medium were transfected at 1e6 cells/ml with 1 ug/ml plasmid DNA and 3 ug/ml linear 25 kDa PEI. A feed with 0.5% (w/v) TN1 peptone was done 24 hours post-transfection. Culture medium were harvested 120 hpt and IGFBP4 constructs were purified by sequential affinity chromatography on TALON and Streptactin-Sepharose (except for the N-term that was only purified by TALON) as previously described Purified material were desalted in PBS on D-Salt Excellulose columns as recommended by the manufacturer. Protein concentration was determined by Bradford against BSA.

### CBP-4 Conjugation to Alexa Fluor 647

80 μl of 1 mM Alexa Fluor 647-NHS in DMSO was added to 0.4 ml of recombinant CBP-4 (0.2 mg/ml) in 100 mM carbonate pH 8.4, and sample was incubated overnight at room temperature. The reaction was stopped with 150 μl of 200 mM ethanolamine pH 8.0. To remove free dye, sample was diluted with 4.5 ml of water and loaded onto 1 ml Co⁺²-Talon Metal Affinity column equilibrated with PBS. The column was exhaustively washed with PBS and CBP-4 eluted with 2 ml of 1 M imidazole in PBS. To remove imidazole from AF647-CBP-4 conjugate, the sample was concentrated to approximately 200 μl on Biomax (M.W. cut-off 5,000), diluted to original volume with PBS and concentrated again. That process of concentration/dilution was repeated three times. Final volume 0.5 ml (0.14 mg/ml). Recovery 86%.

### Confocal Microscopy Studies

HBEC (100000 cell/well in a 24-well format plate) were seeded on human fibronectin- (40 μg/ml) coated cover slips (Bellco Biotechnology) in 400 μl HBEC media and grown until reached 80% confluence. Cells were then washed twice with D-MEM and incubated in D-MEM for 30 min at 37 C. Then, D-MEM was removed and replaced with 250 μl/well of D-MEM containing 100 nM AF647-CBP-4 conjugate for 90 min and then washed with PBS. Cells were counterstained with the membrane dye DiOC₅(3) for 15 seconds and then washed with PBS. Imaging of cells was performed using Zeiss LSM 410 (Carl Zeiss, Thornwood, N.Y., USA) inverted laser scanning microscope equipped with an Argon\Krypton ion laser and a Plan-Apochromat 63X, NA 1.4. Confocal images of two fluoroprobes were sequentially obtained using 488 and 647 nm excitation laser lines to detect DiOC₅(3) (510-525 nm emission) and Alexa 647 fluorescence (670-810 nm emission).

### Results

### DB-cAMP Modulates Proliferation, Invasiveness and Angiogenic Capacity of U87MG Cells

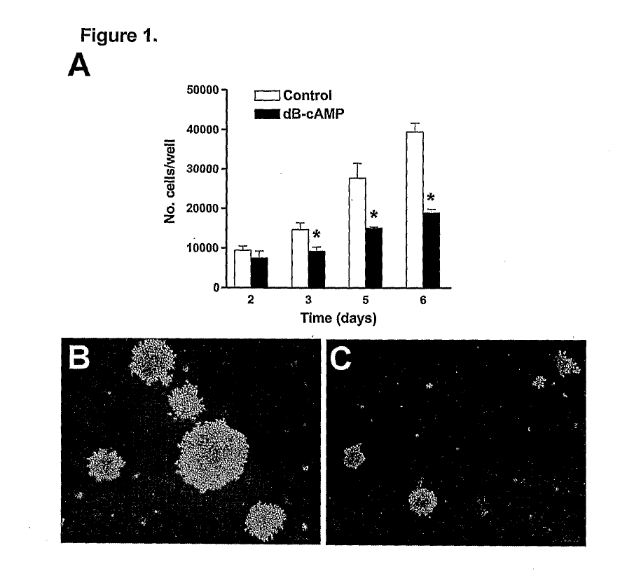

The influence of dB-cAMP on U87MG cell proliferation was determined using CyQuant Cell Proliferation Assay Kit. The proliferation decreased significantly (p<0.05) in dB-cAMP-treated U87MG cells at days 3 (˜30%), 5 (˜45%) and 6 (˜50%) (FIG. 1A). Cell death rates, monitored in the same cultures by in situ staining with propidioum iodide, were not significantly different between dB-cAMP-treated and untreated cells (data not shown). To account for differences in proliferation rates between untreated and dB-cAMP-treated cells, in all subsequent experiments, the number of cells was equalized by adjusting the plating densities.

dB-cAMP-treated U87MG cells also displayed reduced growth in semi-solid agar. Although the total number of colonies formed by untreated and dB-cAMP-treated cells was not significantly (p<0.05) different (data not shown), the size of colonies (total covered area per field) formed was significantly reduced (˜75%) from 1.1±0.3 mm² by untreated cells (FIG. 1B) to 0.3±0.1 mm² by dB-cAMP treated cells (FIG. 1C).

Angiogenic properties of U87MG cells were evaluated on HBEC grown in a mixture of basement membrane components, Matrigel™. This method is widely used to assess angiogenic transformation of peripheral endothelial cells (Nagata et al., 2003) and has been adapted by us (Semov et al., 2005) to evaluate angiogenic responses of brain endothelial cells. HBEC plated in Matrigel™ in D-MEM display a typical spindle-shaped morphology (FIGS. 2A & G) with occasional spiky and elongated cell shapes. When exposed to U87MG CM, HBEC grown in Matrigel™ extended processes that connected into complex tubule-like structures (FIGS. 2B, E & G). BBEC exposed to conditioned media from dB-cAMP-treated U87MG cells failed to form CLT (FIGS. 2C & G). This effect was not due to residual dB-cAMP in CM, since the addition of 500 μM dB-cAMP to U87MG CM did not inhibit CLT formation (FIG. 2D).

The angiogenic response of HBEC induced by U87MG CM was more reproducible (observed in all 7 HBEC preparations studied) than that induced by 20 ng/ml VEGF alone (CLT formation observed in 3 out of 7 HBEC isolations). However, CLT formation induced by U87MG CM was blocked in the presence of the neutralizing VEGF antibody (1 μg/ml) (FIGS. 2F & G). This suggested that, while VEGF is necessary for angiogenic activity of U87MG CM, its effect is most likely potentiated by other angiogenic mediator(s) present in the media.

The levels of the principal pro-angiogenic factors, VEGF, P1GF, IGF-1 and bFGF were determined by ELISA in conditioned media of both U87MG and HBEC cells. VEGF-A levels were 20% higher in CM of dB-cAMP-treated (˜80 ng/ml) U87MG compared to media of untreated (˜60 ng/ml) cells (data not shown). Interestingly, levels of other known angiogenic growth factors, P1GF, IGF-1 and bFGF, were below the detection limit in CM of either untreated or dB-cAMP-treated U87MG (data not shown). Similarly, no detectable release of P1GF, IGF-1 or bFGF was observed in conditioned media of HBEC, with the exception of one HBEC preparation where low levels of bFGF (˜120 pg/ml) were detected by ELISA. Since P1GF, IGF-1 and bFGF were thus ruled out as contributors to angiogenic activity of U87MG CM, the nature of other released angiogenic mediators that are modified by dB-cAMP was investigated using gene microarray approach.

### DB-cAMP Effect on U87MG Gene Expression

To identify molecular correlates of the functional changes described above, differential gene expression between U87MG and U87MG exposed to 500 μM dB-cAMP for 6 days was studied using human 19.2K cDNA glass microarrays.

Scatter plot analysis of normalized fluorescent Cy-3- and Cy-5 signals on microarrays showed that most spots gather around a 45° diagonal line with slope close to 1 and linear regression factor ranging between R²=0.85-0.93 (data not shown). Significant differential gene expression was considered when the normalized mean intensity ratio was >1.5-fold and one sample t-test analysis indicated p<0.05. 55 genes/ESTs were significantly up-regulated (˜1.5-13-fold) and 92 genes/ESTs significantly down-regulated (˜1.5-2.6-fold) in dB-cAMP-treated U87MG cells (Table I and II) by these data selection criteria.

Validation of microarray data was carried out for a selected group of genes (Table III and IV) based on their reported roles in cell differentiation (STC-1 and Wnt-5), growth factor modulation (IGF/IGFBPs/IGFBP proteases) or angiogenesis (PAI-1, SPARC, VEGF).

a) dB-cAMP Induces the Expression of Differentiation-Related Genes

Increased expression of two genes, stanniocalcin-1 (STC-1, 3.43-fold) and Wnt-5A (2.96-fold), both previously implicated in cell differentiation (Wong et al., 2002, Olson and Gibo, 1998) were detected in dB-cAMP-treated U87MG cells by microarray analyses. Q-PCR analysis demonstrated similar levels of up-regulation (STC-1: 3.56-fold and Wnt-5A: 4.03-fold) as those observed by microarray analyses (Table IV).

### b) Validation of Differentially Expressed Angiogenesis-Related Genes by Q-PCR

We hypothesized that the inability of dB-cAMP-treated U87MG CM to induce CLT in HBEC was caused by either a decrease in pro-angiogenic or an increase in anti-angiogenic secreted factors. Using Gene Ontology annotation, we classified the differentially expressed genes based on the cellular localization of their encoded proteins and focused the study on secreted proteins (Table IV). The preponderance of encoded secreted proteins in the up-regulated (12 out of 30 genes) compared to the down-regulated (5 out of 45 genes) group of genes suggested the presence of anti-angiogenic factors in the dB-cAMP-treated U87MG CM.

From the list of genes differentially expressed in dB-cAMP-treated U87MG cells, a group of 6 genes encoding secreted proteins was selected for Q-PCR validation. IGFBP-4, IGFBP-7 and their specific proteases, pregnancy-associated plasma protein-A (PAPP-A) and protease, serine, 11 (IGF binding) (PRSS-11), belong to the IGF growth factor family with multiple functions, including cell growth modulation and tumorigenesis (Zumkeller and Westphal, 2001). Plasminogen activator inhibitor type 1 (PAI-1) and secreted acidic cysteine rich glycoprotein (SPARC) are proteins involved in extracellular matrix (ECM) remodeling and angiogenesis (Stefansson and McMahon, 2003; Brekken and Sage, 2001).

Q-PCR confirmed trend of changes detected by microarray analyses for all genes studied (Table IV). To control for potential false negatives, the expression of IGF-I, IGF-II and IGFBP-3, that showed no change by microarray analyses, was also assessed by Q-PCR. The basal expression levels of IGF-I and -II were low (Ct values ˜31.5-35.0 and ˜35.0-38.0, respectively) compared to ACTB (Ct value ˜16.5-18.5) and no changes were detected in dB-cAMP-treated U87MG (data not shown). IGFBP-3 mRNA levels were not significantly different between dB-cAMP-treated and untreated cells at day 6. (data not shown).

### c) Validation of Differentially Expressed Genes at the Protein Level

Correlation between mRNA expression and protein levels for a select group of genes was investigated by Western-blot, ELISA and enzymatic assays.

mRNA of PAI-1, a serine protease inhibitor prominently involved in ECM turnover and regulation of glioma cell motility and invasion (Hjortland et al., 2003), was up-regulated (microarray: 2.57-fold; Q-PCR: 2.2) at day 6 of dB-cAMP treatment (Table IV). Western blot analysis confirmed up-regulation of PAI-1 protein in dB-cAMP-treated U87MG cells (FIG. 3A). Concurring with increased PAI-1 expression, a 30% reduction in plasminogen activator activity (PAA) was detected in dB-cAMP-treated cells (FIG. 3B). The levels of SPARC (FIG. 3C) and IGFBP-4 (FIG. 3D) measured by ELISA were 5- and 15-fold higher, respectively, in CM of dB-cAMP-treated cells compared to untreated cells.

IGFBP-4 Mediates the Loss of Angiogenic Properties in dB-cAMP-treated U87MG Cells

IGFBP-4, the smallest of the IGFBP members, binds to IGF-I and inhibits IGF-I-induced responses in various cells (Wetterau et al., 1999, Ravinovsky et al., 2002). IGF-I regulates multiple functions such as cellular growth, survival and differentiation under different physiological and pathological conditions (Lopez-Lopez et al., 2004).

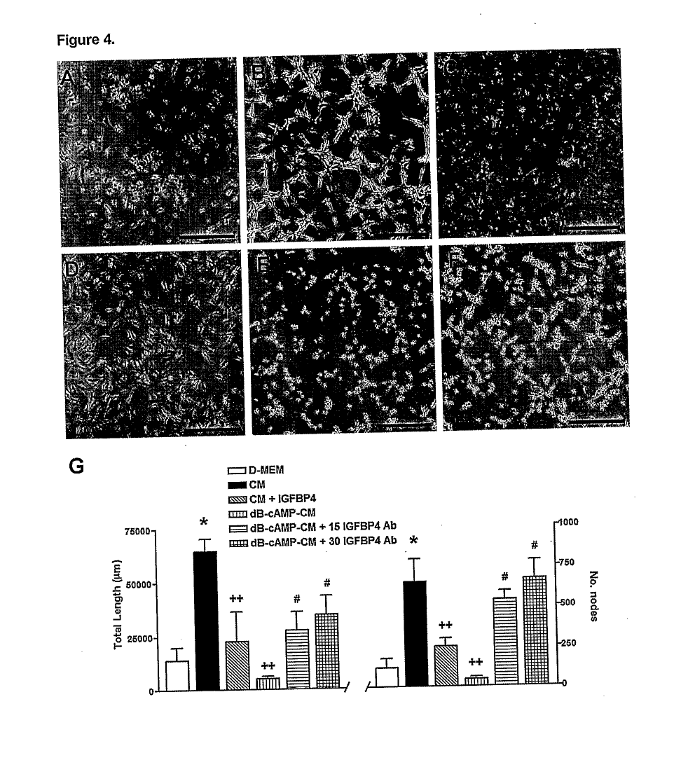

Recombinant IGFBP-4 (500 ng/ml) reversed U87MG CM-stimulated CLT (FIG. 4A-C) to control levels (FIG. 4G). Conversely, anti-IGFBP-4 antibody (20-30 μg/ml), which selectively binds IGFBP-4 and does not cross-react with IGFBP-1, -2 or -3, restored the ability of dB-cAMP-treated U87MG CM to induce CLT in HBEC (FIG. 4D-G). Incubation of HBEC with anti-IGFBP-4 antibody alone did not affect CLT formation (data not shown). These observations strongly suggested that dB-cAMP-stimulated secretion of IGFBP-4 is responsible for the inhibition of angiogenic properties of U87MG.

Recombinant IGFBP-4 (500 ng/ml) potently inhibited IGF-1 (150 ng/ml)-induced CLT formation by HCEC (FIG. 5A). However, since IGF-I was not detectable in either untreated or dB-cAMP-treated U87MG or HBEC, IGFBP-4 anti-angiogenic action against U87MG CM cannot be attributed to direct IGF-I binding. This conclusion is further supported by experiments showing the pleiotropic anti-angiogenic effects of IGFBP-4 against a variety of pro-angiogenic factors including VEGF₁₆₅ (20 ng/ml) (FIG. 5B), P1GF (100 ng/ml) (FIG. 5C) and bFGF (20 ng/ml) (FIG. 5D).

IGFBP-4 (500 ng/ml) also significantly reduced U87MG growth in semi-solid agar (FIG. 6A-B). The treatment reduced the size (from 1.5±0.6 mm² to 0.4±0.3 mm² total area per field), rather than the number, of tumor colonies (FIG. 6A-D). Interestingly, IGFBP-4 (500 ng/ml) did not affect U87MG proliferation rates (data not shown). The anti-tumorigenic effect of IGFBP-4 was pleiotropic since it similarly reduced (˜45%) the size of Hela tumor colonies in a semi-solid agar (FIG. 6E-H)

### Discussion

The results reported in this study suggest that dB-cAMP induces differentiation, reduces proliferation, attenuates invasiveness, and inhibits angiogenic properties of human glioblastoma cells through a coordinated temporal regulation of a subset of genes and proteins involved in cellular differentiation, growth factor modulation, extracellular matrix remodeling and angiogenesis. The inhibition of angiogenesis-inducing properties of U87MG cells by dB-cAMP is a novel finding that may provide insight into mechanisms of cAMP-mediated tumor growth inhibition in vivo (Tortora et al., 1995). The principal mediator of the anti-angiogenic effect was a secreted protein, IGFBP-4, highly expressed in the dB-cAMP-treated U87MG CM. Moreover, IGFBP-4 showed pleiotropic anti-angiogenic and anti-tumorigenic activities, both properties of potential therapeutic relevance for the treatment of glioblastomas and other tumors.

Previous studies (Noguchi et al., 1998; Grbovic et al., 2002) suggested that U87MG growth inhibition by another cAMP analog, 8-Cl-cAMP, results from both G2/M arrest and increased apoptosis. In this study, dB-cAMP reduced U87MG proliferation without affecting viability suggesting that it lacks the cytotoxic effects of adenosine metabolites (Koontz and Wicks, 1980). As reported in other cancer cells (Okamoto and Nakano, 1999), dB-cAMP also reduced the size of colonies formed by U87MG in semi-solid agar. Potential molecular effectors of these actions were mined from differential gene expression data. It is important to note that, given the long stimulation time with dB-cAMP required to produce differentiation and growth suppression effects in U87MG, the differentially expressed gene map reflects the end-point differences in two cellular phenotypes resulting from both direct stimulation of CRE-regulated transcription and secondary effects of stimulated effectors.

The up-regulation of STC-1 and Wnt-5, both previously implicated in cell differentiation (Wong et al., 2002; Olson and Gibo, 1998), suggested that these genes might be downstream effectors of dB-cAMP-induced U87MG differentiation. Up-regulation of STC-1 in parallel with cellular differentiation and neurite outgrowth has recently been described in dB-cAMP-treated neuroblastoma cells (Wong et al., 2002). Wnt-5 is a member of a highly conserved family of growth factors implicated in many developmental decisions, including stem cell control (Walsh and Andrews, 2003) and cell differentiation (Olson and Gibo, 1998).

This is the first study demonstrating loss of angiogenic properties of U87MG glioblastoma cells after exposure to dB-cAMP. In GBM and other tumors with a significant component of necrosis, VEGF is a major inducer of angiogenesis and vasculogenesis (Plate et al., 1992). Inhibition of VEGF production in response to cAMP analogues has been reported in glioblastoma cells (Drabek et al., 2000). In this study, U87MG cells responded to dB-cAMP by a moderate induction (20%) of secreted immunoreactive VEGF-A. This observation suggested that the loss of angiogenic properties of glioblastoma cells treated with dB-cAMP was not caused by reduced VEGF secretion, but rather by mediators capable of counteracting secreted angiogenic factors, including VEGF-A.

Several genes previously shown to modulate angiogenesis were differentially expressed in dB-cAMP-treated U87MG cells. PAI-1 and SPARC modulate angiogenesis through ECM remodeling and were both induced by dB-cAMP. PAI-1, the principal inhibitor of urokinase type plasminogen activator (uPA) and tissue PA (tPA), promotes angiogenesis at low concentrations and inhibits both angiogenesis and tumor growth at high concentrations (Stefansson et al., 2003). SPARC is an ECM-associated glycoprotein with three structural domains implicated in the regulation of proliferation, cell adhesion, ECM synthesis, cell differentiation and angiogenesis (Sage et al., 2003). The effect of SPARC on these processes depends on the nature of the bioactive peptides generated from its cleavage by proteolytic enzymes (Sage et al., 2003).

Several members of the IGF family of growth factors including IGFBP-4, IGFBP-7 and their proteases PAPP-A and PRSS-11 were up-regulated in dB-cAMP-treated U87MG cells. The IGF system includes IGF-I and IGF-II, the type I and type II IGF receptors and specific IGF-binding proteins (IGFBP-1-6). The members of this family have been shown to regulate both normal and malignant brain growth (Hirano et al., 1999). Enhanced expression of IGF-I and IGF-II mRNA transcripts as well as both types of IGF receptors has been associated with aberrant angiogenesis in gliomas (Hirano et al., 1999; Zumkeller, and Westphal, 2001). IGFBPs enhance or inhibit IGF actions by preventing its degradation and modulating its interactions with the receptors (Wetterau et al., 1999). IGFBPs are regulated by post-translational modifications, including phosphorylation, glycosylation, and proteolysis (Wetterau et al., 1999). Both in vitro and in vivo experiments suggest that the IGF system represents an important target for the treatment of malignant central nervous system tumors (Zumkeller and Westphal, 2001).

IGFBP-4, a CREB-regulated gene (Zazzi et al., 1998) and potent inhibitor of IGF-I and tumor proliferation (Zumkeller and Westphal, 2001), was the principal anti-angiogenic mediator secreted by glioblastoma cells in response to dB-cAMP. This conclusion was supported by the following experimental observations: a) IGFBP-4 was significantly up-regulated at both mRNA and protein levels in dB-cAMP-differentiated U87MG cells, b) the addition of recombinant IGFBP-4 blocked U87MG CM-induced angiogenic phenotype in HBEC and c) IGFBP-4 antibody restored angiogenic transformation of brain endothelial cells in response to CM of dB-cAMP-treated U87MG cells. Moreover, IGFBP-4 exhibited a pleiotropic anti-angiogenic action against a variety of pro-angiogenic mediators including VEGF₁₆₅, P1GF, and bFGF.

IGF-I has been shown to promote endothelial cell migration and capillary-like tube formation indirectly by inducing VEGF expression through IGF-IR-activation (Stoeltzing et al., 2003). Neither U87MG nor HBEC cells expressed or secreted detectable levels of IGF-I, suggesting that the anti-angiogenic effect of IGFBP-4 against U87MG-CM is IGF-I independent. This conclusion was further supported by the observation that IGFBP-4 inhibited the angiogenic transformation of brain endothelial cells induced by VEGF₁₆₅, P1GF, and bFGF, none of which has known binding or signaling activity on IGF-IR.

Several IGF-I-independent actions of IGFBP-4 have been demonstrated in other cell systems including a marked inhibition of ceramide-induced apoptosis in Hs578T human breast cancer cells that lack functional IGF-IR (Perks et al., 1999) and modulation of both granulose cell steroidogenesis and CaCo2 human colon cancer cells mitogenesis (Wright et al., 2002; Singh et al., 1994). IGF-I-independent IGFBP-4 actions resulting in inhibition of angiogenic endothelial transformation could involve several potential mechanisms. IGFBP-4 may bind endothelial receptor capable of inhibiting common pro-angiogenic signaling pathways induced by different growth factors; however, no cellular IGFBP-4 receptor has been identified yet, suggesting that IGFBP-4 likely does not trigger a ‘classical’ receptor-mediated signal transduction in endothelial cell. Some IGFBP members have heparin-binding domains (HBD) through which they interact with glycosaminoglycans (Hodgkinson et al., 1994) and modulate IGF-I and potentially other growth factor binding to ECM components, including vitronectin (Kricker et al., 2003); however, IGFBP-4 lacks an HBD and does not GAGs on endothelial cells (Booth et al., 1995). IGFBP-4 may bind directly to other growth factors disrupting their interaction with receptors; this has been reported for IGFBP-3, that binds to latent transforming growth factor beta (TGF-β) binding protein-1 (Gui Y and Murphy; 2003). Interestingly, our unpublished observations suggest that the fluorescently-labeled IGFBP-4 is internalized into HBEC by yet uncharacterized endocytic pathway.

In addition to IGFBP-4, IGFBP-7 and two IGFBP proteases (PRSS11 and PAPP-A) were also induced by dB-cAMP. PAPP-A is a metalloprotease that selectively cleaves IGFBP-4 (Byun et al., 2000). However, its proteolytic activity depends on the presence of IGFs (Qin et al., 2000). Given that U87MG lack detectable IGF-I and express very low levels of IGF-II, cleavage of IGFBP-4 by PAPP-A is not expected in this system. IGFBP-3 and -4 can be degraded to some extent by plasmin and thrombin (Booth et al., 2002), also unlikely in this experimental paradigm since the observed up-regulation of PAI-1 and reduction of plasminogen activator activity suggest reduced plasmin levels.

In addition to inhibiting U87MG-induced angiogenesis, IGFBP-4 also inhibited U87MG and HeLa cell colony formation in semi-solid agar. Overexpression of IGFBP-4 has previously been shown to delay the onset of prostate (Damon et al., 1998) and colorectal (Diehl et al., 2004) colony formation. The observed inhibitory effect of IGFBP-4 on both U87MG tumorigenicity and angiogenesis induced by multiple mediators, suggests that IGFBP-4 could be a pluripotent anti-tumor factor potentially effective in late stage tumors.

In conclusion, dB-cAMP inhibits glioblastoma cell growth and angiogenic competence by inducing a complex program of gene expression involved in cell differentiation, extracellular matrix remodeling, angiogenesis and growth factor modulation. IGFBP-4 was shown to be the principal dB-cAMP-induced anti-angiogenic mediator with strong anti-tumorigenic properties against U87MG cells. Mapping of IGFBP-4 domains involved in these actions will be essential for developing IGFBP-4 analogues with desired anti-angiogenic and anti-tumorigenic functions

While the preferred embodiments of the invention have been described above, it will be recognized and understood that various modifications may be made therein, and the appended claims are intended to cover all such modifications which may fall within the spirit and scope of the invention.

### REFERENCES

The inclusion of a reference is not an admission or suggestion that it is relevant to the patentability of anything disclosed herein.
Booth B A, Boes M, Dake B L, Knudtson K L, and Bar R S. 2002. IGFBP-3 binding to endothelial cells inhibits plasmin and thrombin proteolysis. Am J Physiol Endocrinol Metab. 282 (1):E52-8.
Booth B A, Boes M, Andress D L, Dake B L, Kiefer M C, Maack C, Linhardt R J, Bar K, Caldwell E E, Weiler J, et al. 1995. IGFBP-3 and IGFBP-5 association with endothelial cells: role of C-terminal heparin binding domain. Growth Regul.; 5 (1): 1-17.
Brekken R A, and Sage E H. 2001. SPARC, a matricellular protein: at the crossroads of cell-matrix communication. Matrix Biol. 19(8): 816-27.
Byun D, Mohan S, Kim C, Suh K, Yoo M, Lee H, Baylink D J, and Qin X. 2000. Studies on human pregnancy-induced insulin-like growth factor (IGF)-binding protein-4 proteases in serum: determination of IGF-II dependency and localization of cleavage site. J Clin Endocrinol Metab. 85(1): 373-81.
Cao Y. 2004. Antiangiogenic cancer therapy. Semin Cancer Biol., 14(2): 139-45.
Dalbasti T, Oktar N, Cagli S, and Ozdamar N. 2002. Local interstitial chemotherapy with sustained release bucladesine in de novo glioblastoma multiforme: a preliminary study. J Neurooncol 56(2):167-74.
Damon S E, Maddison L, Ware J L, Plymate S R. 1998. Overexpression of an inhibitory insulin-like growth factor binding protein (IGFBP), IGFBP-4, delays onset of prostate tumor formation. Endocrinology. 139(8):3456-64.
Diehl D, Hoeflich A, Wolf E, Lahm H. Insulin-like growth factor (IGF)-binding protein-4 inhibits colony formation of colorectal cancer cells by IGF-independent mechanisms. Cancer Res. 2004 Mar. 1; 64(5):1600-3.
Ferrara N. 2002. VEGF and the quest for tumour angiogenesis factors. Nat Rev Cancer, 2(10): 795-803.
Gui Y, and Murphy L J. 2003. Interaction of insulin-like growth factor binding protein-3 with latent transforming growth factor-beta binding protein-1. Mol Cell Biochem. 250 (1-2):189-95.
Hodgkinson S C, Napier J R, Spencer G S, and Bass J J. 1994. Glycosaminoglycan binding characteristics of the insulin-like growth factor-binding proteins. J Mol Endocrinol. 13(1): 105-12.
Kim K J, Li B, Winer J, Armanini M, Gillett N, Phillips H S, Ferrara N. 1993. Inhibition of vascular endothelial growth factor-induced angiogenesis suppresses tumour growth in vivo. Nature, 362 (6423): 841-4.
Kricker J A, Towne C L, Firth S M, Herington A C, and Upton Z. 2003. Structural and functional evidence for the interaction of insulin-like growth factors (IGFs) and IGF binding proteins with vitronectin. Endocrinology 144(7):2807-15.
Perks C M, Bowen S, Gill Z P, Newcomb P V, and Holly J M. 1999. Differential IGF-independent effects of insulin-like growth factor binding proteins (1-6) on apoptosis of breast epithelial cells. J Cell Biochem. 75(4):652-64.
Plate K H, Breier G, Weich H A, and Risau W. 1992. Vascular endothelial growth factor is a potential tumour angiogenesis factor in human gliomas in vivo. Nature 359 (6398): 845-8.
Propper D J, Saunders M P, Salisbury A J, Long L, O'Byrne K J, Braybrooke J P, Dowsett M, Taylor M, Talbot D C, Ganesan T S, and Harris A L. 1999. Phase I study of the novel cyclic AMP (cAMP) analogue 8-chloro-cAMP in patients with cancer: toxicity, hormonal, and immunological effects. Clin Cancer Res. 5(7):1682-9.
Singh P, Dai B, Dhruva B, and Widen S G. 1994. Episomal expression of sense and antisense insulin-like growth factor (IGF)-binding protein-4 complementary DNA alters the mitogenic response of a human colon cancer cell line (HT-29) by mechanisms that are independent of and dependent upon IGF-I. Cancer Res. 54(24): 6563-70.
Stanimirovic, D., Morley, P., Ball, R., Hamel, E., Mealing, G., and Durkin, J. P. 1996. Angiotensin II-induced fluid phase endocytosis in human cerebromicrovascular endothelial cells is regulated by the inositol-phosphate signaling pathway. J. Cell. Physiol. 169: 455-467.
Wright R J, Holly J M, Galea R, Brincat M, and Mason H D. 2002. Insulin-like growth factor (IGF)-independent effects of IGF binding protein-4 on human granulosa cell steroidogenesis. Biol Reprod. 67(3):776-81.

TABLE I
Genes up-regulated in dB-cAMP-treated U87MG
cells determined by microarray analyses.

| Approved | Localization (GO | Function | Fold | | p value |
|---|---|---|---|---|---|
| Gene Symbol | Classification) | (GO Classification) | change | SD | (t-test) |
| TFPI2 | Secreted | Serine-type endopeptidase | 13.11 | 2.34 | 0.0091 |
| | | inhibitor | | | |
| TFPI2 | Secreted | Serine-type endopeptidase | 5.27 | 1.77 | 0.0101 |
| | | inhibitor | | | |
| PTGS2 | Membrane | Oxidoreductase | 3.59 | 1.69 | 0.0072 |
| DUSP1 | Cytosol | MAP kinase phosphatase | 3.51 | 1.36 | 0.0038 |
| STC1 | Secreted | Hormone | 3.43 | 1.44 | 0.0066 |
| PRSS11 | Secreted | Chymotrypsin activity | 3.24 | 2.04 | 0.0455 |
| IGFBP4 | Secreted | Growth factor binding | 3.15 | 2.01 | 0.0458 |
| | | protein | | | |
| SMOC1 | ECM | Extracellular matrix | 3.14 | 1.21 | 0.0013 |
| | | component (calcium ion | | | |
| | | binding) | | | |
| WNT5A | Secreted/ECM | Secreted signaling protein | 2.96 | 1.33 | 0.0047 |
| SERPINE 1 | Secreted | Serine protease inhibitor | 2.57 | 1.44 | 0.0142 |
| DUSP1 | Cytosol | MAP kinase phosphatase | 2.43 | 1.31 | 0.0038 |
| PAPPA | Secreted | Endopeptidase | 2.39 | 1.40 | 0.0136 |
| LRP8 | Membrane | Low-density lipoprotein | 2.38 | 1.44 | 0.0173 |
| | | receptor | | | |
| COTL1 | Cytosol | cytoskeleton-related protein | 2.30 | 1.54 | 0.0311 |
| | | (actin-binding protein) | | | |
| CD74 | Membrane | Immune response | 2.24 | 1.04 | 0.0009 |
| TGM2 | Intra and | Acyltransferase activity | 2.09 | 1.14 | 0.0015 |
| | Extracelluar | | | | |
| EPB41L5 | Cytosol/Membrane | Cytoskeleton-plasma | 2.08 | 1.21 | 0.0047 |
| | | membrane interaction | | | |
| SYNPO | Cytosol | Actin-associated protein | 2.04 | 1.18 | 0.0035 |
| HLA-DQA1 | Membrane | MHC class II receptor | 1.96 | 1.18 | 0.0037 |
| PBEF | Secreted | Cytokine-like | 1.96 | 1.17 | 0.0031 |
| SPARC | Secreted/ECM | Matrix-associated protein | 1.88 | 1.46 | 0.0435 |
| NT5E | Membrane | Nucleotide catabolism | 1.87 | 1.27 | 0.0142 |
| HLA-DPB1 | Membrane | MHC class II receptor | 1.83 | 1.20 | 0.0070 |
| AKR1C2 | Cytosol | Oxidoreductase | 1.78 | 1.32 | 0.0255 |
| HLA-DRB3 | Membrane | MHC class II receptor | 1.78 | 1.42 | 0.0458 |
| HLA-DRB1 | Membrane | MHC class II receptor | 1.78 | 1.35 | 0.0314 |
| COL6A1 | ECM | Extracellular matrix | 1.77 | 1.42 | 0.0474 |
| | | structural constituent | | | |
| HNRPD | Nucleus | Regulation of transcription | 1.77 | 1.39 | 0.0400 |
| | | (DNA binding) | | | |
| SPARC | Secreted/ECM | Matrix-associated protein | 1.76 | 1.08 | 0.0055 |
| SPARC | Secreted/ECM | Matrix-associated protein | 1.75 | 1.34 | 0.0318 |
| EREG | Secreted | Growth factor | 1.62 | 1.12 | 0.0031 |
| IGFBP7 | Secreted | Growth factor binding | 1.58 | 1.29 | 0.0363 |
| | | protein | | | |
| DDX5 | Nucleus | ATP dependent helicase | 1.54 | 1.16 | 0.0106 |
| | | activity | | | |
| SFPQ | Nucleus | Nucleic acid binding | 1.51 | 1.10 | 0.0179 |

Only genes with known function are included. Redundant spots appear in the list as different entries to illustrate the reproducibility of the data.
GO = Gene Ontology

TABLE II
Genes down-regulated in dB-cAMP-treated U87MG cells.

| Aproved Gene | Localization (GO | Function | Fold | | p value |
|---|---|---|---|---|---|
| Symbol | Classification) | (GO Classification) | change | SD | (t-test) |
| TAF6 | Nuclear | Transcription initiation factor | 2.66 | 0.71 | 0.03950 |
| ASGR1 | Membrane | Receptor (sugar binding) | 2.40 | 0.79 | 0.00490 |
| CANX | Membrane and ER | Calcium ion binding | 2.37 | 0.90 | 0.00452 |
| CHI3L1 | Extracellular | Extracellular matrix structural | 2.35 | 0.69 | 0.02003 |
| | | constituent | | | |
| PPARBP | Nuclear | Transcription initiation factor | 2.35 | 0.80 | 0.02110 |
| DEPP | Nuclear | Regulation of transcription, DNA- | 2.23 | 0.83 | 0.00314 |
| | | dependent | | | |
| CHRM3 | Membrane | Receptor with ion channel activity | 2.18 | 0.77 | 0.00895 |
| CDC42EP3 | Cytosol | Cytoskeletal regulatory protein | 2.17 | 0.76 | 0.01062 |
| | | binding | | | |
| KCND1 | Membrane | Voltage-gated potassium channel | 2.08 | 0.76 | 0.04550 |
| SPHK2 | Cytosol | Apoptosis inhibitor | 2.08 | 0.66 | 0.04034 |
| ASGR1 | Membrane | Receptor (sugar binding) | 1.99 | 0.68 | 0.03808 |
| LRRFIP1 | Nuclear | Transcription represser | 1.96 | 0.78 | 0.04270 |
| NR4A3 | Nuclear | Transcription factor activity | 1.94 | 0.81 | 0.03206 |
| ZNF217 | Nuclear | Transcription factor activity | 1.90 | 0.79 | 0.01219 |
| DPP6 | Membrane | Dipeptidyl peptidase | 1.83 | 0.73 | 0.03248 |
| ZNF317 | Nuclear | Transcription factor activity | 1.79 | 0.79 | 0.01684 |
| C4BPB | Membrane | Blood coagulation | 1.74 | 0.74 | 0.03498 |
| BTBD3 | Nucleus | Protein binding | 1.74 | 0.75 | 0.03129 |
| CAT | Peroxisomal | Oxidoreductase activity | 1.73 | 0.84 | 0.03112 |
| ACTR8 | Cytosol | Cytoskeleton structural constituent | 1.71 | 0.74 | 0.03716 |
| BIRC6 | Cytosol | Apoptosis inhibitor | 1.71 | 0.91 | 0.00963 |
| NUP160 | Nucleus | Nucleocytoplasmic transporter | 1.70 | 0.83 | 0.01148 |
| FYB | Citosolic and | Protein binding | 1.70 | 0.79 | 0.01956 |
| | nuclear | | | | |
| GNA13 | Membrane | GTP-binding protein | 1.67 | 0.79 | 0.02378 |
| LYN | Membrane | Receptor signaling protein tyrosine | 1.64 | 0.74 | 0.04438 |
| | | kinase activity | | | |
| CGR19 | Secreted | Negative regulation of cell | 1.64 | 0.86 | 0.02974 |
| | | proliferation | | | |
| C11orf21 | Cytosol | Unknown | 1.64 | 0.89 | 0.00396 |
| COX5A | Cytosolic | Electron transport | 1.63 | 0.75 | 0.04274 |
| HPRP8BP | Nuclear | Pre-mRNA splicing factor activity | 1.61 | 0.78 | 0.03243 |
| MTP | ER | Lipid transporter | 1.60 | 0.97 | 0.00169 |
| RPL18 | Cytosolic | Ribosomal structural constituent | 1.60 | 0.77 | 0.03804 |
| HMGN1 | Nuclear | Transcription elongation factor | 1.60 | 0.80 | 0.02601 |
| RPC155 | Nuclear | DNA-directed RNA polymerase | 1.60 | 0.77 | 0.03863 |
| | | activity | | | |
| AOF2 | Mitocondria | Electron transport | 1.60 | 0.86 | 0.00755 |
| IFRG28 | Membrane | | 1.58 | 0.79 | 0.02814 |
| ATF5 | Nuclear | Transcription corepressor activity | 1.54 | 0.77 | 0.04747 |
| ACP5 | Lysosomal | Acid phosphatase | 1.52 | 0.79 | 0.03517 |
| TOR2A | Secreted | | 1.52 | 0.78 | 0.04528 |
| MMP12 | Secreted | Matrix-degrading enzyme | 1.51 | 0.86 | 0.01168 |
| | | (proteolysis and peptidolysis) | | | |
| PSG1 | Secreted | Immunomodulation | 1.57 | 0.80 | 0.02842 |
| ATP2B4 | Membrane | Calcium-transporting ATPase | 1.56 | 0.93 | 0.00891 |
| | | activity | | | |
| ACP5 | Lysosomal | Acid phosphatase activity | 1.54 | 0.78 | 0.04211 |
| GDF11 | Secreted | Growth factor | 1.52 | 0.79 | 0.03517 |
| PDE4DIP | Golgi/centrosome | Transcription regulation DNA- | 1.52 | 0.97 | 0.00006 |
| | | dependent | | | |
| ACOX3 | Peroxisomal | Oxidoreductase activity | 1.51 | 0.87 | 0.00868 |

Only genes with known function are included. Redundant spots appear in the list as different entries to illustrate the reproducibility of the data
GO = Gene Ontology,
ER = Endoplasmic reticulum

TABLE III

Primers designed to amplify selected genes
differentially expressed in db-cAMP-treated U87MG cells

| | | | | Amplicon |
|---|---|---|---|---|
| | | | | length |
| Gene | Acc. No. | Forward primer (5′-3′) | Reverse primer (5′-3′) | (bp) |
| IGFBP-4 | XM 04993 | CCC ACT CCC AAA GCT CAG | TGC AAC AAC CAG ACC TAA | 76 |
| IGFBP-7 | NM 00155 | GCG AGC AAG GTC CTT CCA | GGG ATT CCG ATG ACC TCA CA | 93 |
| PRSS-11 | NM 00277 | CCC AAA GGT CAA TGC ACA | CAA CTT CGG CCG TTT GAG AA | 63 |
| PAPPA | NM 00258 | GAC CCA CAT CCC TTT GGT | CCG GTT GGG TGC TAA GGA | 85 |

Each set of forward and reverse primers was designed from NCBI published sequences corresponding to the provided accession number (Acc. No.) using the Primer Express Software v2.0.

TABLE IV
Functional description and comparative analyses of changes observed
by microarray and Q-PCR analyses for the selected group of genes.

| | | Microarray | Q-PCR |
|---|---|---|---|
| Gene | Function | (fold-change) | (fold-change) |
| IGFBP-4 | Protein that binds IGF ligands and prevents their access to | 3.15 | 8.08 |
| | cell surface receptors. | | |
| IGFBP-7 | Member of the IGFBP family of proteins. May function as | 1.58 | 1.52 |
| | a growth suppressing factor. | | |
| PAPP-A | Metalloprotease that selectively cleaves IGFBP-4. | 2.39 | 7.81 |
| PRSS-11 | A serine protease specific for IGF binding proteins | 3.24 | 2.82 |

TABLE V

INSULIN-LIKE GROWTH FACTOR BINDING
PROTEIN 4 PROTEIN SEQUENCE INFORMATION

SEQ ID NO. 1
IBP4_HUMAN Insulin-like growth factor binding
protein 4 precursor (IGFBP-4) (IBP-4) (IGF-binding
protein 4) - Homo sapiens (Human) (aas 1-258)
MLPLCLVAALLLAAGPGPSLGDEAIHCPPCSEEKLARCRPPVGCEELVRE
PGCGCCATCALGLGMPCGVYTPRCGSGLRCYPPRGVEKPLHTLMHGQGVC
MELAEIEAIQESLQPSDKDEGDHPNNSFSPCSAHDRRCLQKHFAKIRDRS
TSGGKMKVNGAPREDARPVPQGSCQSELHRALERLAASQSRTHEDLYIIP
IPNCDRNGNFHPKQCHPALDGQRGKCWCVDRKTGVKLPGGLEPKGELDCH
QLADSFRE
Sequences of the IGFBP4 constructs generated to
identify the protein region containing the anti-
angiogenic activity.
Details:
SSP-IGFBP4 (Full length)-SH8Q1, 1 to 258, Draw as
Gene Translation product 282 aas Mol Wt 30782.5,
Isoelectric Pt (pI) 6.83
SEQ ID NO. 2
Translation:
MLPLCLVAALLLAAGPGPSLGDEAIHCPPCSEEKLARCRPPVGCEELVRE
PGCGCCATCALGLGMPCGVYTPRCGSGLRCYPPRGVEKPLHTLMHGQGVC
MELAEIEAIQESLQPSDKDEGDHPNNSFSPCSAHDRRCLQKHFAKIRDRS
TSGGKMKVNGAPREDARPVPQGSCQSELHRALERLAASQSRTHEDLYIIP
IPNCDRNGNFHPKQCHPALDGQRGKCWCVDRKTGVKLPGGLEPKGELDCH
QLADSFREVDPWSHPQFEKTGHHHHHHHHGGQ
Details:
SSP-IGFBP-4 (N-term)-SH8Q1, aa 1 to 156, Draw as
Gene Translation product 179 aas Mol Wt 19348.8,
Isoelectric Pt (pI) 6.57
SEQ ID NO. 3:
Translation:
MLPLCLVAALLLAAGPGPSLGDEAIHCPPCSEEKLARCRPFVGCEELVRE
PGCGCCATCALGLGMPCGVYTPRCGSGLRCYPPRGVEKPLHTLMHGQGVC
MELAEIEAIQESLQPSDKDEGDHPNNSFSPCSAHDRRCLQKHFAKIRDRS
TSGGKMDPWSHPQFEKTGHHHHHHHHGGQ
Details:
SSP-IGFBP4 (C-term)-SH8Q1, aa 155 to 258, Draw as
Gene Translation product 146 aas Mol Wt 16334.3,
Isoelectric Pt (pI) 7.74
SEQ ID NO. 4
Translation:
MLLLLLLLGLRLQLSLGIASKVNGAPREDARPVPQGSCQSELHRALERLA
ASQSRTHEDLYIIPIPNCDRNGNFHPKQCHPALDGQRGKCWCVDRKTGVK
LPGGLEPKGELDCHQLADSFREVDPWSHPQFEKTGHHHHHHHHGGQ
Red: Signal Peptide (SSP)
Blue: Streptag-II/(His)₈G tag (SH8Q1)
IGFBP-4 domain located in the C-terminal region
Details:
Thyroglobulin type-I domain (aas 200249)
SEQ ID NO. 5
Translation:
P IPNCDRNGNF HPKQCHPALD GQRGKCWCVD

TABLE VI

CLUSTAL W (1.83) multiple sequence
alignment of IGFBP tyroglobulin type-1 domains.

| | |
|---|---|
| IGFBP-3 | HIPNCDKKGFYKKKQCRPSKGRKRGFCWCVD-KYGQPLPGYTTKGKEDVHC |
| IGFBP-5 | YLPNCDRKGFYKRKQCKPSRGRKRGICWCVD-KYGMKLPGMEYVDG-DFQC |
| IGFBP-6 | YVPNCDHRGFYRKRQCRSSQGQRRGPCWCVD-RMGKSLPGSPDGNG-SSSC |
| IGFBP-1 | YLPNCNKNGFYHSRQCETSMDGEAGLCWCVYPWNGKRIPGSPEIRG-DPNC |
| IGFBP-2 | HIPNCDKHGLYNLKQCKMSLNGQRGECWCVNPNTGKLIQGAPTIRG-DPEC |
| IGFBF-4 | PIPNCDRNGNFHPKQCHPALDGQRGKCWCVDRKTGVKLPGGLEPKG-ELDC |
| | :***::.* :. :**. : . . * **** * : * . * |

1. (canceled)

2. (canceled)

3. (canceled)

4. (canceled)

5. (canceled)

6. (canceled)

7. (canceled)

8. Use of a peptide comprising 20 or more consecutive amino acids of amino acids 1 to 258 of SEQ ID No. 1 in the preparation of a medicament for inhibiting angiogenesis.

9. The use according to claim 8 wherein the peptide comprises amino acids 200-249 of SEQ ID No. 1.

10. The use according to claim 8 wherein the peptide comprises amino acids 155-268 of SEQ ID No. 1.

11. The use according to claim 8 wherein the peptide comprises amino acids 1-165 of SEQ ID No. 1.

12. Use of a peptide comprising at least 85% identity to amino acids 200-249 of SEQ ID No. 1 in the preparation of a medicament for inhibiting angiogenesis.

13. Use of a peptide comprising at least 85% identity to amino acids 1-156 of SEQ ID No. 1 in the preparation of a medicament for inhibiting angiogenesis.

14. Use of a peptide comprising at least 85% identity to amino acids 157-258 of SEQ ID No. 1 in the preparation of a medicament for inhibiting angiogenesis.

15. Use of a peptide comprising 20 or more consecutive amino acids of amino acids 1 to 258 of SEQ ID No. 1 in the preparation of a medicament for inhibiting tumor growth.
